SEQUENCE LISTING (8 sequences)

SEQ ID NO: 1 - DNA, 20 bp, Artificial Sequence
  artificially synthesized sequence
gagtcaacggatttggtcgt

SEQ ID NO: 2 - DNA, 20 bp, Artificial Sequence
  artificially synthesized sequence
gacaagcttcccgttctcag

SEQ ID NO: 3 - DNA, 19 bp, Artificial Sequence
  artificially synthesized sequence
ggtacagtgcagggaaaga

SEQ ID NO: 4 - DNA, 20 bp, Artificial Sequence
  artificially synthesized sequence
cttgccacacaatcatcacc

SEQ ID NO: 5 - DNA, 20 bp, Artificial Sequence
  artificially synthesized sequence
ggcacatcaagcagccatgc

SEQ ID NO: 6 - DNA, 22 bp, Artificial Sequence
  artificially synthesized sequence
tagttcctgctatgtcacttcc

SEQ ID NO: 7 - DNA, 20 bp, Artificial Sequence
  artificially synthesized sequence
cctcagccattacacaggcc

SEQ ID NO: 8 - DNA, 20 bp, Artificial Sequence
  artificially synthesized sequence
ccttggtgggtgctactcct

(19) United States
(12) Patent Application Publication
(10) Pub. No.: US 2014/0107015 A1
(43) Pub. Date: Apr. 17, 2014

(54) PHARAMACEUTICAL COMPOSITION FOR PREVENTING OR TREATING HUMAN IMMUNODEFICIENCY VIRUS

(71) Applicant: Pukyong National University Industry-University Cooperation Foundation, Busan (KR)

(72) Inventors: Se-Kwon KIM, Busan (KR); Fatih KARADENIZ, Busan (KR)

(73) Assignee: Pukyong National University Industry-University Cooperation Foundation, Busan (KR)

(21) Appl. No.: 13/675,240

(22) Filed: Nov. 13, 2012

(30) Foreign Application Priority Data
Oct. 11, 2012 (KR) 10-2012-0112956

(51) Int. Cl. C07H 13/04; C07K 9/00

(52) U.S. Cl. CPC C07H 13/04; C07K 9/001
USPC 514/3.8; 536/53; 514/55

(57) ABSTRACT

There is provided a pharmaceutical composition for preventing or treating human immunodeficiency virus, and more particularly, to a pharmaceutical composition and health functional food for preventing or treating/improving human immunodeficiency virus, the pharmaceutical composition and health functional food including a new compound with chitooligosaccharides conjugated amino acids or dipeptides as an effective component. The new compound has an excellent anti-HIV effect through an activity of inhibiting a HIV initial infection by interrupting an interaction between host-virus membranes, and also activities of inhibiting reverse transcriptase and protease of HIV. The compound according to the present invention is a conjugate synthesized through conjugating chitooligosaccharides derived from a natural material with amino acids or dipeptides. The compound is stable without cytotoxicity.

### CROSS-REFERENCE TO RELATED APPLICATIONS

This application claims the priority of Korean Patent Application No. 2012-0112956 filed on Oct. 11, 2012 in the Korean Intellectual Property Office, the disclosure of which is incorporated herein by reference.

### BACKGROUND OF THE INVENTION

1. Field of the Invention

The present invention relates to a pharmaceutical composition for preventing or treating human immunodeficiency virus, and more particularly, to a pharmaceutical composition and health functional food for preventing or treating human immunodeficiency virus, the pharmaceutical composition and health functional food including a new compound with chitooligosaccharides conjugated amino acids or dipeptides as an effective component.

2. Description of the Related Art

After an AIDS patient was first reported in 1981, it was confirmed by isolating human immunodeficiency virus (HIV), AIDS virus from HIV was important pathogen that causes AIDS.

HIV belongs to retrovirus, taxonomically, and among them, is subdivided into lentivirus. A diameter of HIV particle is approximately 10 micron, the outside of HIV is surrounded by phospholipid like a general cell membrane, and two virus genomes composed of RNAs inside the HIV is protected in the a core protein (capsid). Such genomes are composed of 10 genes that mean to have many genes as compared with the whole size of genomes.

A HIV infection is accomplished by conjugating an envelope protein (gp120) on the virus surface and receptor on the surface of the target cells. The receptor is a cell surface protein molecular called CD4 antigen. The main target of HIV is a CD4 cell (helper T cell) including many such CD4 antigens or macrophage on the cell surface. The phospholipid of virus is fused to the cell surface by conjugating between such virus and cells, and virus genomes and nucleus protein enter in a cell. At this time, RNA of the virus genomes is changed into DNA by reverse transcriptase present in the virus particles, transferred in cell nucleus, and then inserted into a genome of the host cell. Such a process is one of retrovirus properties. While hiding in a most stable place inside host cells as described above, HIV is supported by all the mechanisms and sources required for a growth from cells. In addition, HIV can protect itself and survive from an immune body, and the like while suppressing or stimulating its growth according to the situations and conditions.

AIDS virus is largely classified into two types, such as HIV-1 and HIV-2. Since HIV-1 is found in patients in many countries including Korea, HIV-1 is thought to be the epitome of AIDS virus. HIV-2 is mainly found in patients in West Africa, the base sequence of genome of HIV-2 is equal to only about 55% of the base sequence of genome of HIV-1 and rather is more similar to that of Simian Immunodeficiency Virus (SIV), monkey AIDS virus. It is known that toxicity of HIV-2 is largely weak as compared with that of HIV-1. HIV varies genetically and also biologically. The base sequences of HIVs isolated from many AIDS patients are different from each other, and also even in cases of HIVs isolated from the same patient, the base sequences of virus are different from each other according to the progress of the disease. Even in a case of HIVs isolated from the same patient at a particular time, the viruses having different base sequences are detected according to the tissue sites. Such diverse base sequences have been closely associated with the determination of various biological properties of virus. Virus having different base sequence has different infection preference to a specific cell, proliferation rate, a virus production amount, toxicity to cells, a forming rate of multinucleated giant cells, incubation period and activity abandonment, sensitivity to neutralizing antibody, and the like. According to the results of researching about interrelation between such various biological properties and AIDS outbreak up to now, virus isolated from an initial patient does not usually form multinucleated giant cells (NSI: Nonsyncytia-Inducing) and preferentially infects microphages. However, as AIDS progressed to the end of AIDS, more and more the forming ability (SI; Syncytia-Inducing) of multinucleated giant cells increases and is changed into the virus that preferentially infects helper T cells instead of the macrophages. It suggests that the biological properties of HIV are relevant to pathogenesis.

Virus can be easily detected in bloods of patients as virus is actively proliferated at approximately a week after infected with HIV. Such a stage is called viremia. Such virus sharply decreases, so that the virus is less likely to be isolated within 1 to 2 weeks. The virus is maintained in such an incubation period for a long time, and then again the virus is actively proliferated as AIDS is developed to be in a state of viremia. Recently, as a result of research worked by using polymerase chain reaction, it was reported that virus is continuously produced even in an incubation period, and thus such a report has been noticed. The number of CD4 cells suddenly decreases during first viremia, but again is recovered in a certain value as the proliferation of virus decreases (Health people: 500 to 1000 CD4 cells/mm³). Since then, the number of CD4 cells gradually decreases over years. When the number of CD4 cells decreases at 200 CD4 cells per 1 mm³ of blood or less, ARC (AIDS-related Complex) or AIDS is developed. Since AIDS patients have a high opportunistic infection opportunity, so that they will die from pheumocysitis carinii pneumonia, and the like. A period of sharply decreasing the proliferation of HIV, in which the proliferation is increased at the beginning of the infection, corresponds with a period of increasing CD8 cells. It is known that CD8 cells function as suppressing a growth of cell or selectively killing the virus-infected cells. Accordingly, it is considered that CD8 cells exhibit an important immune effect for initial infected virus. Antibody is produced after decreasing virus as a proper time. There are CD8 cells, antibodies, and the like from the beginning of the infection to the onset of AIDS, but their functions are lost or modified, and even they may stimulate the virus infection. It is a problem we must solve that how are they lost their immune systems, but they distinctly have anti-virus effects at the beginning of the infection. Since AIDS has the distinct characteristics that AIDS occurs only in human, the understanding of the causes of HIV is still in the beginning stage immensely. All scholars agree that the decrease of CD4 cells directly causes an immune deficiency. However, there are a number of differing views about that how does HIV decrease CD4 cells. As the explanation of such a decrease, various theories, such as the formation of multinucleated giant cells, the accumulation of virus DNA that are not inserted, the influence of host cell membrane structure, an abandonment of programmed cell apoptosis, a secretion of toxicity from the infected cells, and a cytoclasis due to autoimmunity, are suggested. However, there is nothing to verify the fact that occurs actually in vivo. There are many researches about a process of HIV occur using monkey and monkey AIDS virus SIV. However, no remarkable findings were announced.

There is Zidovudine (AZT) suppressing the function of HIV reverse transcriptase as AIDS medicine that is one of the most widely used now. Thus, the studies about the effectiveness of AZT have been done. However, when using AZT at the beginning of the infection, it can improve the clinical condition in some degree, but it cannot affect on extending the life of patient. That is, there are side effects, such as toxicity to bone marrow, and when using it for a long period of time, virus that developed resistance to it is produced. Therefore, there is a limit to use it as a medicine. In cases of The Food and Drug Administration approved DDI, DDC, d4T, and the like as a drug having the same function as AZT, the virus is becoming resistant to them also. However, the drugs are less toxic than AZT.

Above this, many methods for suppressing the growth of virus are developed, and their performances are verifying. For example, there are a method for inhibiting virus from being attached to cells, a method for selectively killing only the virus-infected cells, a method for using a drug (for example, protease, integrase, tat inhibitor, rev inhibitor) of suppressing the function of enzyme that is important for a growth of virus, a method using cytokines, a method for injecting CD8 cells, a genetic treatment method, and the like. There are many progresses for developing HIV vaccine. Very various methods, such as a method for using killed virus or attenuated virus, a method using a specific protein of virus that is expressed using a genetic engineering technology (subunit vaccine), and a method directly injecting anti-idiotype antibody, DNA, and gene, are developing. However, a fundamental problem for developing vaccine is that viruses vary excessively. For example, after a protective inoculation, the growth of virus was suppressed by antibody when using original virus that was used for developing vaccine, but was not suppressed completely by antibody when using the infected cells or viruses that were newly collected from a patient. Such diversity of virus to be overcome remains an unsolved problem.

Accordingly, a development of AIDS treatment drug capable of replacing by solving the problems, such as side effects of such AIDS treatment drugs and an appearance of resistance virus, is urgently needed.

Meanwhile, chitin and chitosan are a naturally presented polysaccharide polymer, and is receiving attention as a new functional material in the recent years. A chemical structure of chitin is polysaccharide polymer bound with many N-acetylglucosamine (β-1,4 conjugating) having N-acetylamide (—NHCOCH₃) as a substituent instead of —OH group at C-2 site of glucopyranose of cellulose. Chitosan is produced by deacetylating an acetyl group of chitin.

Chitosan is very useful biomaterial produced from a crustacean such as shrimp and crab, and its chemical name is called (1→4)₂-amino-2-deoxy-β-D-glucosamine. Such chitosan has various physiological activities as it is, such as effects on lowering of blood cholesterol and inhibiting of fat absorption, anti-tumor effect, effect on improving immunity, antidiabetic effect, effect on treating wound, and anti-bacterial effect. Especially, since chitosan exhibits effect on stimulating absorption of drug by conjugating to mucilage layer, such as oral cavity, the nasal cavity, and intestine cavity, use of chitosan as an absorption enhancer are being actively researched for various oral dosage forms of protein drugs or non-absorbent drugs.

Especially, chitooligosaccharides (COS) (hereinafter, referred to as ‘COS’) is a chitosan derivative (polyvalent cation polymer including glucosamine unit), and may be produced by chemically or enzymatically hydrolyzing chitosan. COS is non-toxic and a component having high solubility. Since COS exhibits various physiological activities such as an anti-bacterial effect, anti-cancer effect, anti-oxidative effect, effect on suppressing angiotension-converting enzyme, effect on boosting immunity, it receiving great attention from the recent medical and pharmaceutical fields.

Accordingly, the present inventors paid attention to chitooligosaccharides (COS) while searching a material that is capable of exhibiting an excellent effect on preventing or treating human immunodeficiency virus (HIV) and also is harmless to humans because of deriving from a natural material; synthesized a new compound (conjugate) produced by conjugating chitooligosaccharide with amino acid or dipeptides; and tested anti-HIV activity of the new compound. As a result, the present inventors confirmed that the new compound (conjugate) according to the present invention has an activity of suppressing HIV infection by interrupting the interaction between host-virus membranes, and also has an excellent anti-HIV activity through suppressing protease and reverse transcriptase of HIV. Therefore, the present inventors completed the present invention.

### SUMMARY OF THE INVENTION

An aspect of the present invention provides an effective pharmaceutical composition for preventing or treating human immunodeficiency virus (HIV), the composition being harmless to humans.

An aspect of the present invention also provides an effective HIV inhibitor for preventing or treating human immunodeficiency virus (HIV), the inhibitor being harmless to humans.

An aspect of the present invention also provides effective health functional food for preventing or improving human immunodeficiency virus (HIV), the health functional food being harmless to humans.

An aspect of the present invention also provides a method of preventing or treating human immunodeficiency virus (HIV) infection.

In order to achieve the above-described objects, according to an aspect of the present invention, there is provided a pharmaceutical composition for preventing or treating human immunodeficiency virus (HIV), the composition including a compound represented by the following Chemical Formula 1 as an effective component:

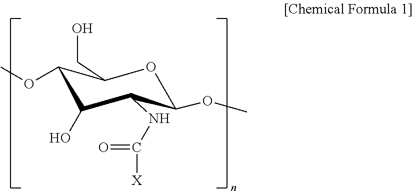

(where,

X is selected from the group consisting of Chemical Structures listed in the following Table; and

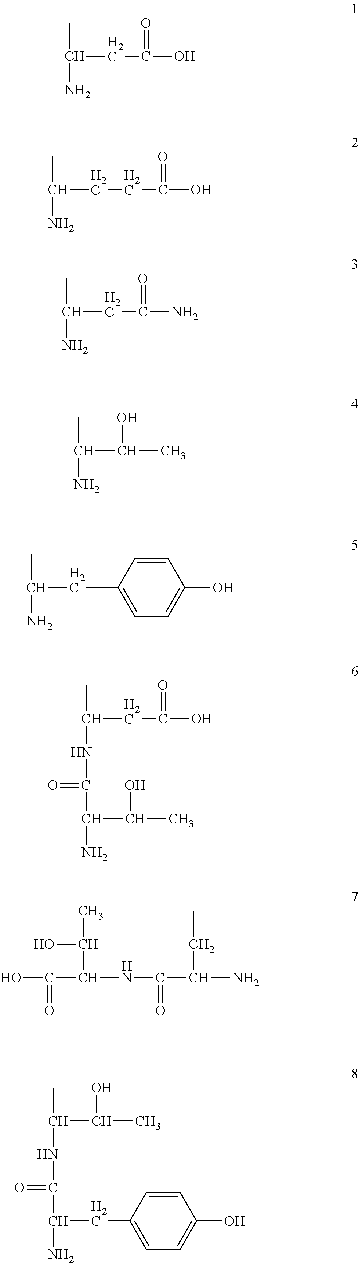

n is an integer of 2 to 6.)

According to an embodiment of the present invention, the composition including the compound represented by Chemical Formula 1 (here, x is Chemical Structure 1 or 5) as an effective component may have an anti-HIV effect through an activity of inhibiting reverse transcriptase of HIV.

According to an embodiment of the present invention, the composition including the compound represented by Chemical Formula 1 (here, x is Chemical Structure 3 or 4) as an effective component may have an anti-HIV effect through an activity of inhibiting a HIV infection by interrupting an interaction between host-virus membranes.

Also, according to the present invention, the composition including the compound represented by Chemical Formula 1 (here, x is Chemical Structure 2) as an effective component may have an anti-HIV effect through an activity of inhibiting protease of HIV.

According to an embodiment of the present invention, the composition including the compound represented by Chemical Formula 1 (here, x is one selected from Chemical Structures 6 to 8) as an effective component may have an anti-HIV effect through activities of inhibiting reverse transcriptase of HIV and a HIV infection by interrupting an interaction between host-virus membranes.

According to an embodiment of the present invention, the compound represented by Chemical Formula 1 described above may be included in a concentration of 0.01 to 10 mg/ml in the composition.

Also, the present invention provides a HIV inhibitor including the composition described above as an effective component.

Also, the present invention provides health functional food for preventing or improving human immunodeficiency virus (HIV), the health functional food including the compound represented by Chemical Formula 1 described above as an effective component.

According to an embodiment of the present invention, the health functional food including the compound represented by Chemical Formula 1 (here, x is Chemical Structure 1 or 5) as an effective component may have an anti-HIV effect through an activity of inhibiting reverse transcriptase of HIV.

According to an embodiment of the present invention, the health functional food including the compound represented by Chemical Formula 1 (here, x is Chemical Structure 3 or 4) as an effective component may have an anti-HIV effect through an activity of inhibiting a HIV infection by interrupting an interaction between host-virus membranes.

According to an embodiment of the present invention, the health functional food including the compound represented by Chemical Formula 1 (here, x is Chemical Structure 2) as an effective component may have an anti-HIV effect through an activity of inhibiting protease of HIV.

According to an embodiment of the present invention, the health functional food including the compound represented by Chemical Formula 1 (here, x is one selected from Chemical Structures 6 to 8) as an effective component may have an anti-HIV effect through activities of inhibiting reverse transcriptase of HIV and a HIV infection by interrupting an interaction between host-virus membranes.

According to an embodiment of the present invention, the food may be selected from the group consisting of drinks, meats, chocolates, foods, confectionery, pizzas, ramen, other noodles, rice cakes, gums, candies, ice creams, alcohol drinks, liquor, vitamins, and health supplements.

Also, the present invention provides a method of preventing or treating human immunodeficiency virus (HIV) infection comprising administering to a subject in need thereof a pharmaceutical composition comprising a compound represented by the Chemical Formula 1 as an effective component.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 represents structures, properties, and abbreviations of 20 amino acids;

FIG. 2 is a schematic diagram illustrating a proposed synthesis mechanism of amino acid conjugated chitosan oligomers;

FIG. 3 is ¹H-NMR spectrum of COS-D according to the present invention (NMR Solvent: DMSO-d₆);

FIG. 4 is ¹³C-NMR spectrum of COS-D according to the present invention (NMR Solvent: DMSO-d₆);

FIG. 5 is ¹H-NMR spectrum of COS-E according to the present invention;

FIG. 6 is ¹³C-NMR spectrum of COS-E according to the present invention;

FIG. 7 is ¹H-NMR spectrum of COS-N according to the present invention;

FIG. 8 is ¹³C-NMR spectrum of COS-N according to the present invention;

FIG. 9 is ¹H-NMR spectrum of COS-T according to the present invention;

FIG. 10 is ¹³C-NMR spectrum of COS-T according to the present invention;

FIG. 11 is ¹H-NMR spectrum of COS-Y according to the present invention;

FIG. 12 is ¹³C-NMR spectrum of COS-Y according to the present invention;

FIG. 13 is ¹H-NMR spectrum of COS-TD according to the present invention;

FIG. 14 is ¹³C-NMR spectrum of COS-TD according to the present invention;

FIG. 15 is ¹H-NMR spectrum of COS-TY according to the present invention;

FIG. 16 is ¹³C-NMR spectrum of COS-TY according to the present invention;

FIG. 17 is graphs illustrating cell survivabilities with time after infecting C8166 cell line with HIV-1_{IIIB} in which the cells were treated with each of 20 conjugates of chitosan oligomers-amino acids of the present invention at the concentration of 0.5 mg/ml;

FIGS. 18 and 19 are microscopic images illustrating a degree of forming syncytia observed with a microscope in C8166 cell lines infected with HIV-1_{RF}, in which the cells were treated with each of 20 conjugates of chitosan oligomers-amino acids of the present invention at the concentration of 0.5 mg/ml;

FIG. 20 is a graph illustrating cell survivability with time in C8166 cell lines infected with HIV-1_{IIIB}, in which the cells were treated with 6 conjugates of chitosan oligomers-amino acids (COS-D, COS-E, COS-G, COS-N, COS-T, COS-Y) and 2 conjugates of chitosan oligomers-dipeptides (COS-TD, COS-TY) according to the present invention at the concentration of 0.5 mg/ml;

FIG. 21 is microscopic image illustrating a degree of forming syncytia observed with a microscope in C8166 cell lines infected with HIV-1_{RF}, in which the cells were treated with 2 conjugates of chitosan oligomers-dipeptides of the present invention at the concentration of 0.5 mg/ml;

FIG. 22 is a graph illustrating the number of syncytia formed at the time of passing 2 days after infecting C8166 cell lines with HIV-1_{RF}, in which the C8166 cell lines were treated with each of 7 conjugates (COS-D, COS-E, COS-N, COS-T, COS-Y, COS-TD, and COS-TY) of the present invention at the concentration of 0.5 mg/ml;

FIG. 23 is a graph illustrating HIV-1 reverse transcriptase inhibiting activity of each of 7 conjugates (COS-D, COS-E, COS-N, COS-T, COS-Y, COS-TD, and COS-TY) of the present invention;

FIG. 24 is a graph illustrating HIV-1 protease inhibiting activity of each of 7 conjugates (COS-D, COS-E, COS-N, COS-T, COS-Y, COS-TD, and COS-TY) of the present invention;

FIG. 25 is a graph illustrating HIV-1 protease inhibiting activity of each of 7 conjugates (COS-D, COS-E, COS-N, COS-T, COS-Y, COS-TD, and COS-TY) of the present invention in HIV-1_{saq} that is saquinavir resistant strain;

FIG. 26 is a graph illustrating HIV-1 reverse transcriptase inhibiting activity in COS-D of the present invention with various concentrations (0.05, 0.1, 0.25, 0.5, and 1 mg/ml);

FIG. 27 is a graph illustrating HIV-1 reverse transcriptase inhibiting activity in COS-Y of the present invention with various concentrations (0.05, 0.1, 0.25, 0.5, and 1 mg/ml);

FIG. 28 is a graph illustrating a rate of inhibiting syncytia formation (% of control) according to the treatment of COS-N of the present invention with various concentrations (0.2, 0.4, 0.6, 0.8, and 1.0 mg/ml);

FIG. 29 is a graph illustrating a rate of inhibiting syncytia formation (% of control) according to the treatment of COS-T of the present invention with various concentrations (0.2, 0.4, 0.6, 0.8, and 1.0 mg/ml);

FIG. 30 is a graph illustrating HIV-1 protease inhibiting activity according to the treatment of COS-E of the present invention with various concentrations (0.05, 0.1, 0.25, 0.5, and 1 mg/ml);

FIG. 31 is a graph illustrating a degree of inhibiting p24 production, in which the p24 is present in culture supernatant after incubating H9 cell infected with HIV-1_{IIIB} for 5 days after treating with COS-E of the present invention with various concentrations to the H9 cells infected with HIV-1_{IIIB};

FIG. 32 is a graph illustrating a rate of inhibiting syncytia formation (% of control) according to COS-TD of the present invention with various concentrations (0.2, 0.4, 0.6, 0.8, and 1.0 mg/ml) in uninfected C8166 cell lines co-cultured with H9 cells infected with HIV-1_{IIIB};

FIG. 33 is a graph illustrating a rate of inhibiting syncytia formation (% of control) according to COS-TY of the present invention with various concentrations (0.2, 0.4, 0.6, 0.8, and 1.0 mg/ml) in uninfected C8166 cell lines co-cultured with H9 cells infected with HIV-1_{IIIB};

FIG. 34 is images illustrating a western blot analysis of p24 protein amount in cells treated with COS-TD and COS-TY of the present invention with various concentrations (0.05, 0.1, 0.25, 0.5, and 1 mg/ml);

FIG. 35 is images illustrating a RT-PCR analysis of HIV-1 gene (Vif, Env, Gag) expression amount according to the treatment of COS-TD and COS-TY of the present invention with various concentrations (0.05, 0.1, 0.5, and 1 mg/ml);

FIG. 36 is a graph illustrating the number of syncytia formation with time after treating COS-TD and COS-TY of the present invention at the concentration of 0.5 mg/ml in the co-culture of 8E5 and MOLT-4 cell lines;

FIG. 37 is a graph illustrating p24 antigen production amount in the infected cells by a delayed addition of COS-TD and COS-TY of the present invention after infecting CEM-SS cells with HIV-1; and

FIG. 38 is a graph illustrating HIV-1 host genome replication inhibiting ability of each of COS-TD and COS-TY of the present invention by a gene reporter assay.

### DETAILED DESCRIPTION OF THE PREFERRED EMBODIMENT

The present invention provides a composition for preventing or treating (improving) human immunodeficiency virus, the composition including a compound represented by the following Chemical Formula 1 as an effective component:

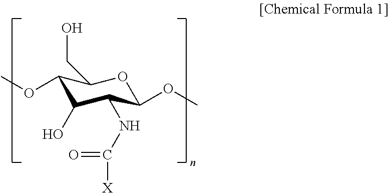

(where,

X is selected from the group consisting of Chemical Structures listed in the following Table; and

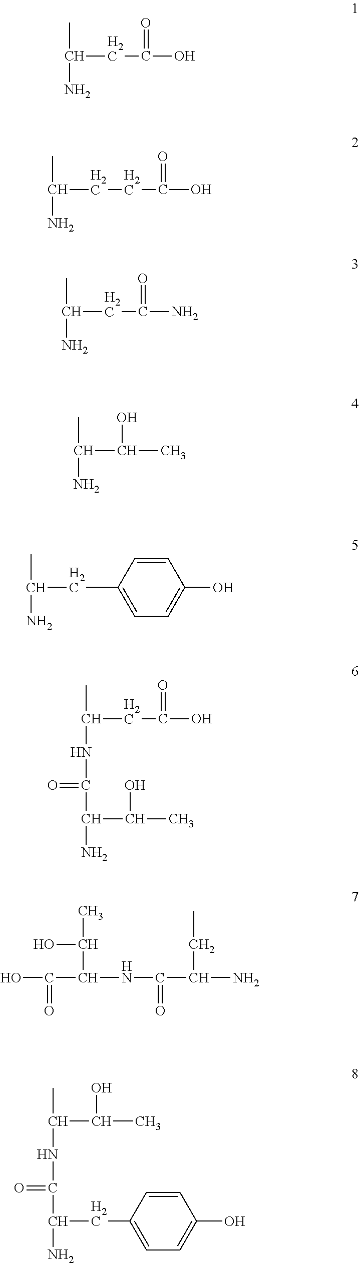

n is an integer of 2 to 6.)

The compound represented by Chemical Formula 1 described above according to the present invention may be synthesized by conjugating chitosan oligomers with amino acid or dipeptides via a series of processes.

According to an embodiment of the present invention, the compound represented by Chemical Formula 1 described above may be obtained by the method including; protecting an alcohol group of chitosan oligomer; synthesizing a conjugate of chitosan oligomer-amino acids or dipeptides through reacting the alcohol group-protected chitosan oligomer with Boc-protected amino acids or dipeptides; and gradually removing THP-ether and Boc protecting group from the conjugate.

The present inventors synthesized the total 22 conjugates of chitosan oligomer-amino acid and chitosan oligomer-dipeptide according to the above-described method (see Table 1), selected 7 conjugates having high anti-HIV activity from them (see Table 2), and named COS-D (x: Chemical Structure 1), COS-E (x: Chemical Structure 2), COS-N (x: Chemical Structure 3), COS-T (x: Chemical Structure 4), COS-Y (x: Chemical Structure 5), COS-TD (x: Chemical Structures 6 and 7), and COS-TY (x: Chemical Structure 8) to each of them.

Especially, the present inventors found that the above-described 7 conjugates can inhibit a HIV initial infection through interrupting an interaction between host-virus membranes, and also can exhibit an excellent anti-HIV effect through inhibiting HIV reverse transcriptase and protease. Accordingly, the present inventors first investigated that the 7 conjugates are effective for preventing or treating (improving) human immunodeficiency virus (HIV).

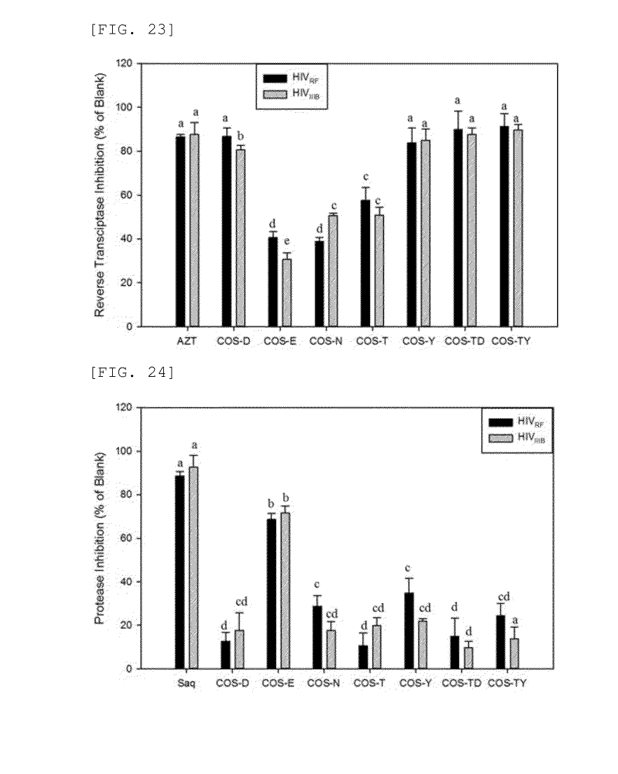

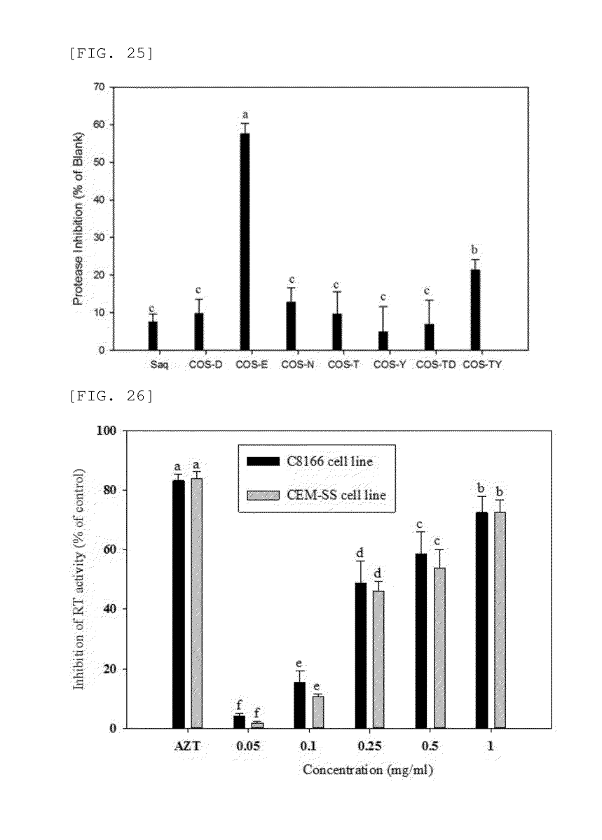

More particularly, according to the following Example 3 of the present invention, it was found as a result of investigating an anti-HIV activity mechanism of the above-described 7 conjugates (COS-D, COS-E, COS-N, COS-T, COS-Y, COS-TD, and COS-TY) that they had an excellent effect on inhibiting cell fusion at the initial stage of virus infection (see FIG. 22) through confirming that COS-D and COD-Y had a high reverse transcriptase inhibiting activity (see Table 3 and FIG. 23), COS-E had a high protease inhibiting activity (FIGS. 24 and 25), and COD-N and COD-T effectively inhibited a syncytia formation. In addition, it was found from the experiment that they had effects of COS-TD and COS-TY on inhibiting virus initial infection and reverse transcriptase at the same time (see Table 4).

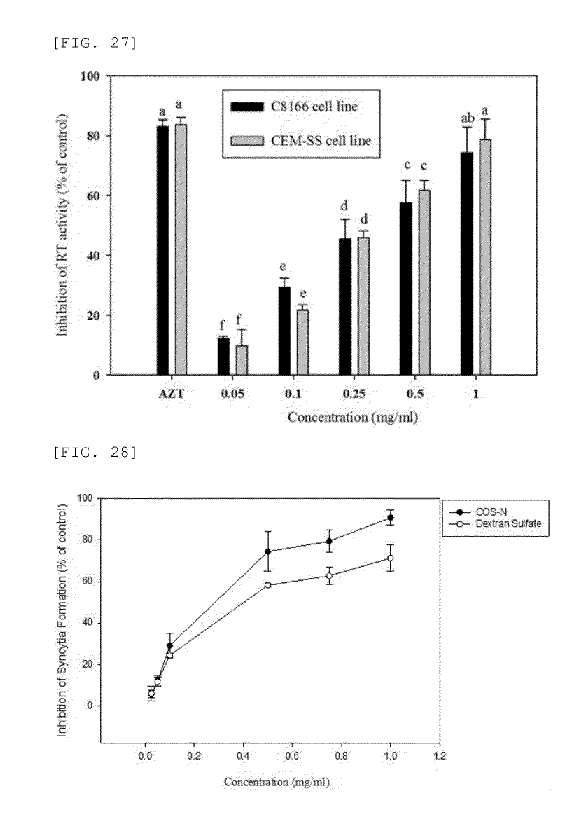

Furthermore, according to the following Examples <4-1> and <4-2>, it was found as a result of investigating HIV reverse transcriptase inhibiting activity according to COS-D and COS-Y of the present invention with various concentrations (0.05, 0.1, 0.25, 0.5, and 1 mg/ml) that each of COS-D and COS-Y of the present invention inhibited HIV reverse transcriptase depending on the concentration (see FIGS. 26 and 27).

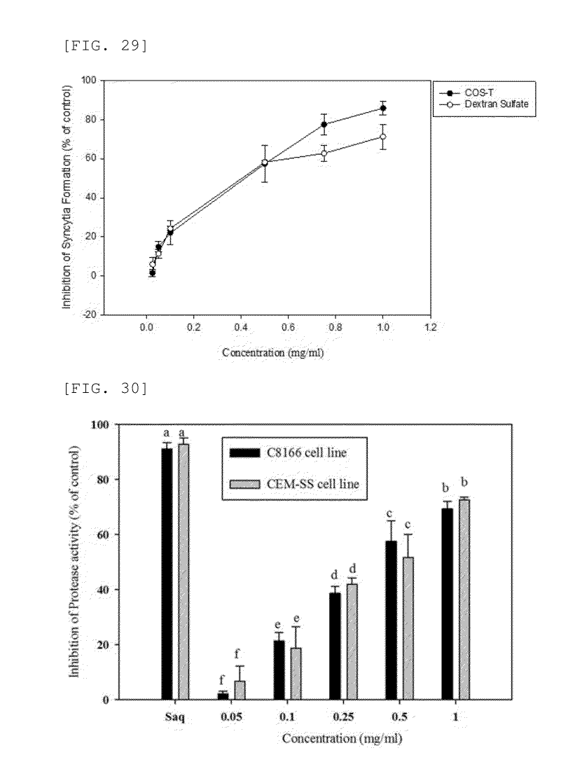

In addition, according to the following Examples <4-3> and <4-4>, it was found as a result of investigating a syncytia formation-inhibiting activity according to each of COS-N and COS-T of the present invention with various concentrations (0.2, 0.4, 0.6, 0.8, and 1 mg/ml) that each of COS-N and COS-T of the present invention inhibited a syncytia formation depending on the concentration in the experimental group treated with the sample (see FIGS. 28 and 29).

In addition, according to the following Example <4-5>, it was found as a result of investigating HIV-1 protease inhibiting activity according to COS-E of the present invention with various concentrations (0.05, 0.1, 0.25, 0.5, and 1 mg/ml) that COS-E of the present invention inhibited HIV-1 protease depending on the concentration (see FIG. 30).

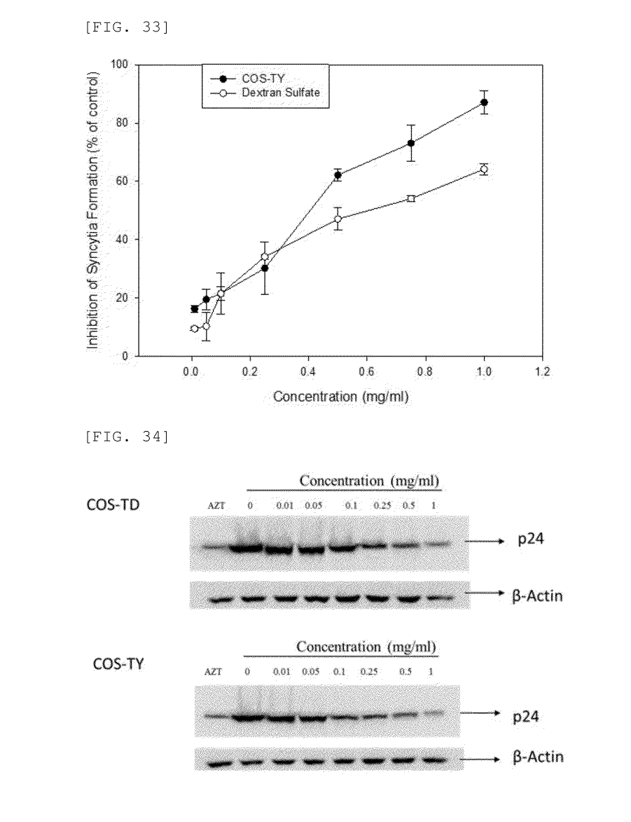

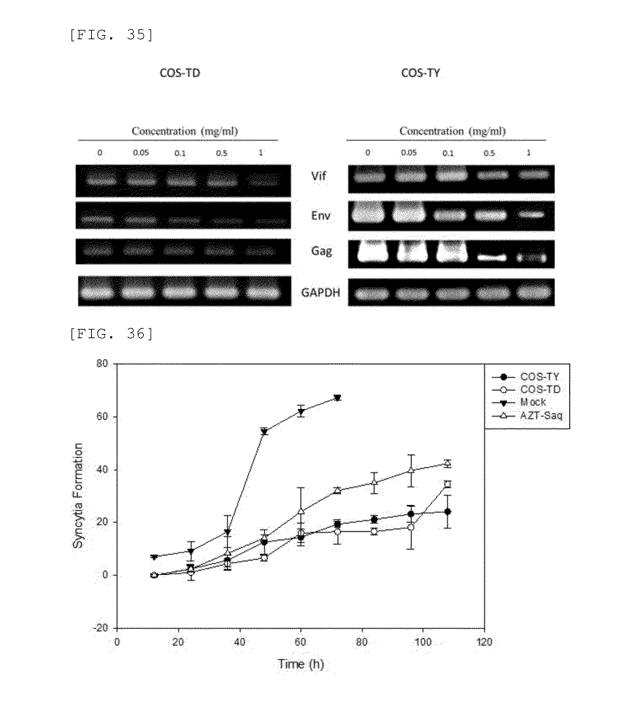

In addition, according to the following Example 5, it was found as a result of investigating an anti-HIV activity according to COS-TD and COS-TY of the present invention with various concentrations that each of COS-TD and COS-TY of the present invention inhibited a syncytia formation depending on the concentration in the experimental group treated with sample (see FIGS. 32 and 33); inhibited p24 antigen production (see FIG. 34); decreased HIV-1 gene (Vif, Env, Gag) expression amount (see FIG. 35), and inhibited HIV-1 host genome replication (see FIG. 38).

From the above-described results, the present inventors verified experimentally that each of the 7 conjugates (COS-D, COS-E, COS-N, COS-T, COS-Y, COS-TD, and COS-TY) had an anti-HIV activity through various mechanisms. Especially, we verified experimentally that since COS-TD and COS-TY of the present invention prevented HIV-1 from being conjugated with a host cell through an interaction with a host cell, and simultaneously, inhibited HIV-1 genome replication through inhibiting HIV-1 reverse transcriptase activity, the COS-TD and COS-TY were effective for preventing or treating (improving) HIV.

For the present invention, the 7 conjugates described above are represented by the compound of Chemical Formula 1 described above. Accordingly, the new compound represented by Chemical Formula 1 according to the present invention has the same meaning as that of the above-described 7 conjugates.

The composition of the present invention may be a pharmaceutical composition or food composition.

The pharmaceutical composition of the present invention may be prepared by using a pharmaceutically suitable and physiologically acceptable adjuvant in addition to the effective component. The adjuvant may include excipient, a disintegrating agent, a sweeting agent, a binding agent, a coating agent, a swelling agent, a lubricant, a modifier, a flavoring agent, and the like.

The pharmaceutical composition may be preferably formulated into a pharmaceutical composition including further at least one pharmaceutically acceptable carrier in addition to the above-described effective component in order for an administration.

A dosage form of the pharmaceutical composition may include granules, powders, tables, coated tables, capsules, suppository, solutions, syrups, juices, suspensions, emulsions, drops, injections, or the like. For example, in order to formulate in a type of tables or capsules, the effective component may be combined with an oral and non-toxic pharmaceutically acceptable inactive carrier, such as ethanol, glycerol, and water. In addition, if necessary or desired, a suitable binding agent, lubricant, disintegrating agent, and color coupler may be included in a mixture. The suitable binding agent may include natural sugar, such as starch, gelatin, glucose, or beta-lactose, natural and synthetic gum, such as a corn sweeting agent, acacia, tragacanth, or sodium oleate, sodium stearate, magnesium stearate, sodium benzoate, sodium acetate, sodium chloride, and the like, but the present invention is not limited thereto. The disintegrating agent may include starch, methyl cellulose, agar, bentonite, xanthan gum, and the like, but the present invention is not limited thereto. For the composition to be formulated into solution, the pharmaceutically acceptable carrier may include, as things that are sterilized or suitable for a living body, saline solution, distilled water, Ringer's solution, buffer saline solution, albumin injection solution, dextrose solution, malto dextrin solution, glycerol, ethanol, and a mixture in combination with at least one of them, and if necessary, other general additives, such as antioxidant, buffer solution, and bacteristat may be added. In addition, diluents, a distributing agent, surfactant, a binding agent, and a lubricant may be further added to formulate it into injective dosage form, such as aqueous solution, suspensions, and emulsion, pills, capsules, granules, and tables. Furthermore, it may be preferably formulated according to each diseases or components using a method disclosed in Remington's Pharmaceutical Science, Mack Publishing Company, Easton Pa. as a proper method in the prior art.

According to an embodiment of the present invention, the compound represented by the above-described Chemical Formula 1 of the present invention may be included in a concentration of 0.01 to 10 mg/ml in the composition and 0.1 to 95 wt % relative to the total weight of the composition.

The composition of the present invention may be oral-administrated or non-oral-administrated (for example, an intravenous injection, an intradermal injection, an intra-abdominal injection, or a topical injection) according to the desired method, and the dosage may be controlled by various factors such as age of a patient, a body weight of a patient, a general health condition of a patient, sex of a patient, diets, an administration time, an administration route, an excretion rate, and a severity of disease.

The composition of the present invention may be used alone, or in combination with the methods of using surgery, hormone treatment, drug treatment, and biological reaction controller in order to prevent or treat AIDS.

The composition of the present invention may be also a food composition. The food composition may include various flavoring agents, natural carbohydrates, or the like as an additive component like the general food composition in addition to the compound represented by the above-described Chemical Formula 1 as an effective component.

An example of the above-described natural carbohydrates may include general sugar, such as monosaccharide, for example, glucose, fructose, and the like; disaccharide, for example, maltose, sucrose, and the like; and polysaccharide, for example, dextrin, cyclodextrin, and the like; and sugar alcohol such as xylitol, sorbitol, and erythritol. As the above-described flavouring agents, a natural flavouring agent (Thaumatin), Stevia extract (for example, Rebaudioside A, Glycyrrhizine, and the like), and a synthetic flavouring agent (saccharine, aspartame, and the like) may be advantageously used.

The food composition according to the present invention may be used as functional foods or may be added to various foods by formulating using the same method as the pharmaceutical composition. As foods, in which the composition of the present invention can be included, there are for example, drinks, meats, chocolates, foods, confectionery, pizzas, ramen, other noodles, gums, candies, ice creams, alcohol drinks, vitamins, health supplements, and the like.

In addition, such a food composition may include various nutrients, vitamins, minerals (electrolyte), tastes such synthetic tastes and natural tastes, coloring agents and enhancers (cheese, chocolates, and the like), pectic acid and a salt thereof, alginic acid and a salt thereof, organic acid, protective colloid thickeners, pH adjuster, stabilizers, preservatives, glycerin, alcohols, a carbonating agent for a carbonated drink, and the like, in addition to the compound represented by the above-described Chemical Formula 1, that is an effective component. In addition to that, the food composition of the present invention may include fruit flesh for preparing natural fruit juices, fruit juice drinks, and vegetable drinks.

In addition, the present invention provides a health functional food for preventing or improving human immunodeficiency virus (HIV), the food including the composition represented by the above-described Chemical Formula 1 as an effective component.

The health functional food of the present invention may be prepared and processed in a type of tablets, capsules, powder, granules, liquids, pills, and the like for preventing and improving human immunodeficiency virus (HIV).

The term, “health functional foods” disclosed in the present invention relates to foods that are prepared and processed by using a raw material or component having useful functionality to humans under the laws about health functional foods, and means foods that are ingested for controlling nutrients for structures and functions of human body and obtaining a useful effect for health care, such as a physiological effect.

The health functional foods of the present invention may include general food additives, and whether or not the health function foods are suitable as a food additive material is determined based on a standard and criteria relating to a relevant item according to general rules disclosed in Korean Food Additives Codex and a general test method that have been approved by Korea Food & Drug Administration as long as other rules do not provide.

The items disclosed in such “Korean Food Additives Codex” may include, for example, a chemically synthetic composite, such as ketones, glycine, calcium citrate, nicotinic acid, and cinnamic acid; a natural additive material, such as persimmon color, licorice extract, microcrystalline cellulose, Kaoliang color, and guar gum; and mixed formulations, such as sodium L-glutamate formulation, alkali agents for noodles, preservative formulation, and tar color formulation.

For example, the health functional foods in a type of tablets may be prepared by granulating a mixture of the compound represented by the above-described Chemical Formula 1 that is an effective component of the present invention with excipient, a binding agent, a disintegrating agent, and other additives through a general method, and then compression-molding through adding a modifier, and the like, or directly compression-molding the mixture described above. In addition, the health functional foods in a type of tablets may include flavor enhancers, and the like if necessary.

Among the health functional foods in a type of capsules, a hard capsule of the health functional foods may be prepared by filling the mixture mixing the compound represented by the above-described Chemical Formula 1 that is an effective component of the present invention with additives such as excipient into a general hard capsule. A soft capsule of the health functional foods may be prepared by filling the mixture mixing the compound represented by the above-described Chemical Formula 1 with additives such as excipient into a capsule basic material such as a gelatin. The soft capsule formulation may include a plasticizer such as glycerin or sorbitol, a coloring agent, preservatives, and the like if necessary.

The health functional foods in a type of a pill may be prepared by molding the mixture mixing the compound represented by the above-described Chemical Formula 1 that is an effective component of the present invention with excipient, a binding agent, and a disintegrating agent by using a known method in the prior art, and if necessary, may be coated with white sugar or other coating agents. In addition, the health functional foods in a type of a pill may be coated with a material such as starch or talc.

The health functional foods in a type of a granule may be prepared by preparing the mixture mixing the compound represented by the above-described Chemical Formula 1 that is an effective component of the present invention with excipient, a binding agent, and a disintegrating agent in a type of a granule by using a known method in the prior art, and if necessary, may include fragrance ingredients, flavor enhancers, and the like.

The health functional foods may be drinks, meats, chocolates, foods, confectionery, pizzas, ramen, other noodles, gums, candies, ice creams, alcohol drinks, vitamins, health supplements, and the like.

In addition, the present invention provides a use of the composition including the compound represented by the above-described Chemical Formula 1 as an effective component for preparing a medicine or food for preventing or treating (improving) human immunodeficiency virus (HIV). The composition including the compound represented by the above-described Chemical Formula 1 as an effective component according to the present invention may be used for preparing a medicine or food for preventing and treating (improving) human immunodeficiency virus (HIV).

In addition, the present invention provides a method for preventing or treating human immunodeficiency virus (HIV), the method including administrating the compound represented by the above-described Chemical Formula 1 to a mammal.

The term “mammal” used in the present specification refers to a mammal that is an object to be treated, observed, or experimented, and preferably human.

The term “therapeutically effective amount” used in the present specification means an amount of an effective component or pharmaceutical composition inducing a biological or medical reaction to a tissue system, animal, or human to be considered by researchers, veterinarians, doctors, or other clinical instructions, and refers to an amount inducing an alleviation of symptoms of diseases or disorders to be treated. The therapeutically effective dosage or dosage number of the effective component according to the present invention may be obvious by the skilled person in the prior art. Accordingly, the optimum dosage to be administrated may be easily determined by the skilled person in the prior art, and may be controlled by various factors such as a type of a disease, severity of a disease, the contents of an effective component and other components included in the composition, a type of dosage form, age of a patient, a body weight of a patient, a general health condition of a patient, sex of a patient, diets of a patient, an administration time, an administration route, a rate of secreting the composition, a treatment period, and drugs to be co-administrated.

According to the treatment method of the present invention, the composition including the compound represented by the above-described Chemical Formula 1 as an effective component may be administrated in a general route, such as an oral administration, a rectum administration, an intravenous injection, an intra-abdominal injection, a topical injection, an intradermal injection, and an inhale route.

Exemplary embodiments of the present invention will now be described in detail with reference to the accompanying drawings.

### EXAMPLE

### Reagent

Chitosan oligomer (having a molecular weight of 1 kDa or less) was supplied from KITTOLIFE; Dihydropyran (DHP), p-Toluene sulfonic acid (pTSA), Hunig's base ((iPr)₂NEt), Hydrochloric acid (aqueous form), tert-butoxycarbonyl (Boc-) protected amino acids, dipeptides, and Amberlyst H15 were purchased from Sigma Chemical Co. (St. Louis, Mo., USA); and Benzene (PhH), Tetrahydrofuran (THF), Methanol (MeOH), 1-Ethyl-3-(3-dimethylaminopropyl)carbodiimide (EDC) were purchased from Junsei Chemical (Tokyo, Japan). All the reagents having an analysis grade were used.

3-(4,5-dimethylthiazol-2-yl)-2,5-diphenyltetrazolium bromide (MTT) reagent and dimethyl sulfoxide (DMSO) were purchased from Sigma Chemical Co. (St. Louis, Mo., USA); RPMI 1640 medium, penicillin/streptomycin, and fetal bovine serum (FBS) required for the cell culture were purchased from Gibco BRL; H9, H9/HIV-1_{IIIB}, MOLT-4, and 8E5 cell lines were purchased from American Type of Culture Collection (Manassas, Va., USA); and CEM-SS cell lines and C8166 cell lines were supplied from Dr. P. Nara and G. Farrar at EU Programme EVA center, respectively.

Cell Culture

H9, H9/HIV-1_{IIIB}, CEM-SS, Jurkat, 8E5 and MOLT-4 and C8166 cell lines were propagated at 37° C. under 5% CO₂ incomplete RPMI 1640 medium supplemented with 10% FBS, 100 μg of streptomycin per ml and 100 U of penicillin per ml. All cells were cultured in either T25 or T75 cell culture flasks. Cells were sub-cultured 2-3 times a week to yield a final concentration of 1×10⁵ cells.

### Example 1

### Synthesis of Chitosan Oligomers Conjugated with Each of Amino Acids or Dipeptides

The present inventors synthesized a chitosan oligomer-amino conjugate acid and a chitosan oligomer-dipeptide conjugate.

<1-1> Synthesis of Chitosan Oligomer-Amino Acids Conjugate

Firstly, chitosan oligomers alcohol groups were protected by tetrahydropyranyl (THP-) ethers derived from the reaction of hydroxyl and dihydropyran. Basically, 0.4 g (8.4 mmol of —NH₂) of chitosan oligomer (below 1 kDa) have been introduced to DHP (4.0 eq.) in PhH, reflux under the mildly acidic catalysis of pTSA. Stirring of reaction mixture for 8 h was followed by the centrifuge at 3,000 rpm for 10 min. Next, unprotected chitosan oligomers were removed by intense MeOH washing for 3 times. Excess DHP was then removed by a rotary evaporator (BUCHI Labortechnik, Switzerland).

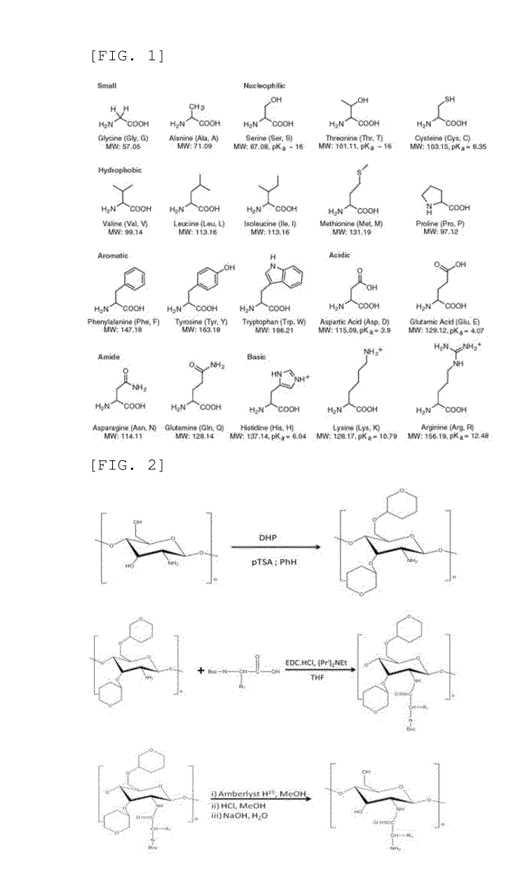

THP-ether protected chitosan oligomers then conjugated by 20 natural amino acids (see FIG. 1) to form a peptide bond between —NH₂ chain of the chitosan oligomer and the carboxyl end of the Boc-protected amino acid. Basically, protected COS from the first step of synthesis was stirred with four equivalents of Boc-protected amino acid in THF (where the amino acids are insoluble in THF, dimethylformamide—DMF was used). For the activating agent of amide bonds, EDC.HCl (1-Ethyl-3-(3-dimethyllaminopropyl)carbodiimide hydrochloride) was used along Hunig's base as the base for the reaction. Mixture was stirred for 12 h and then centrifuged at 3,000 rpm for 10 min. Next, reaction mixture was washed against excess water for 3 times.

Lastly, THP-ether and Boc protections were removed to yield amino acid conjugated chitosan oligomers. Firstly, THP-ether protection was removed by acidic alcoholysis catalyzed by Amberlyst H¹⁵ form in MeOH to give a pH range of 2.0˜3.0. Prior to removing excess solvent including deprotected DHP by rotary evaporator, reaction mixture was centrifuged at 3,000 rpm for 10 min. Next, in order to remove the Boc group, samples were stirred with 1M HCl in MeOH for 4 h. Obtained mixture finally was removed from excess solvent by rotary evaporator and suspended in water and centrifuged at 3,000 rpm for 10 min in order to remove Boc group. Reaction mixture then washed with 1M NaOH solution three times for the purpose of basification. The resulting solution was cooled then dialyzed exhaustively against distilled water using an electrodialyzer (Micro Acilyzer G3, Asahi Chemical Industry Co., Tokyo, Japan), and lyophilized. FIG. 2 illustrates brief conjugation mechanism of the chitosan oligomer-amino acids conjugate according to the present invention.

The chitosan oligomer-amino acids conjugate thus obtained was gained by freeze-drying and purified through a column chromatography using silica (Si) gel 60 (70-230 mesh, Merck, Germany) followed by evaporation of the solvent (MeOH:H₂O 1:1). Final samples were obtained as light brown-yellow powder. Each sample was named by combining the abbreviation of chitosan oligomer (COS) and capital letter abbreviation of conjugated amino acids, and used as a sample for the following experiments.

<1-2> Synthesis of Chitosan Oligomer-Dipeptides Conjugate

The present inventors tried a synthesis of chitosan oligomer-dipeptides conjugate. The method for synthesizing was the same as the method for conjugating the amino acid of Example <1-1> to chitosan oligomer, and Boc-protected dipeptide was used instead of amino acids.

However, only two dipeptides (TD and TY) were able to be conjugated to COS

<1-3> Analysis of Synthesis

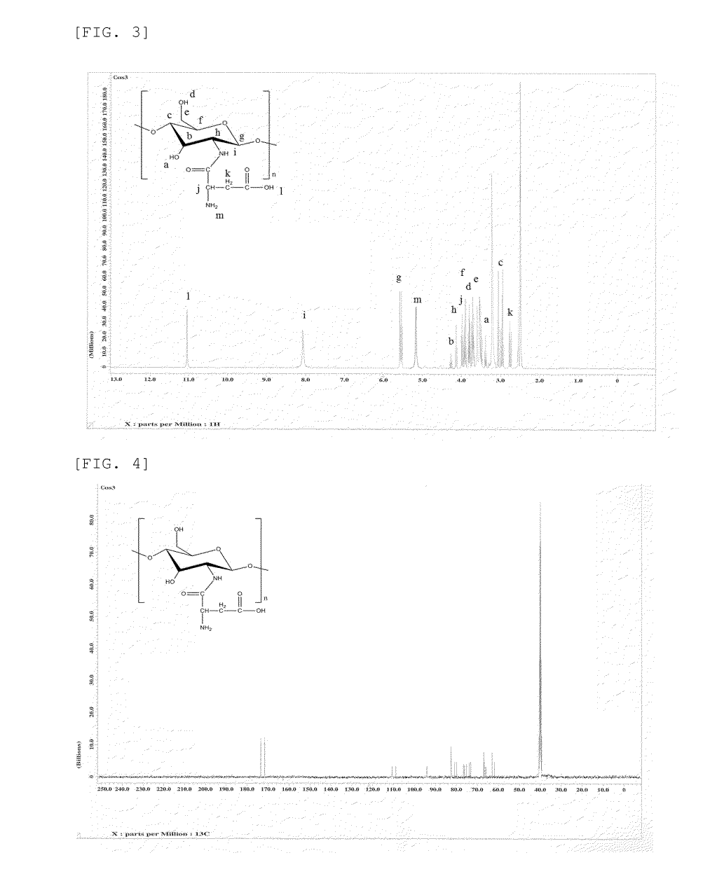

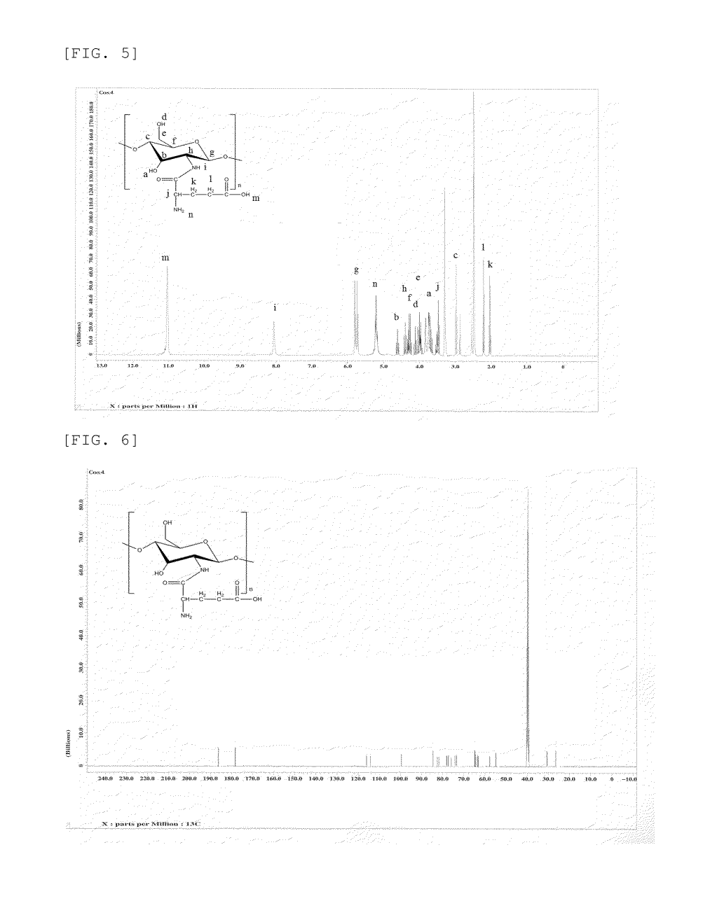

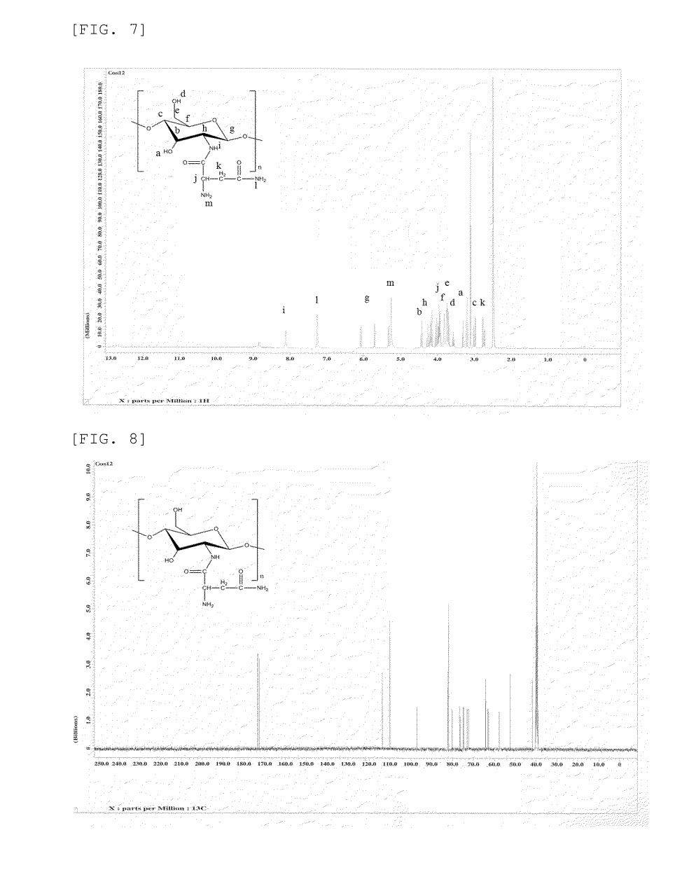

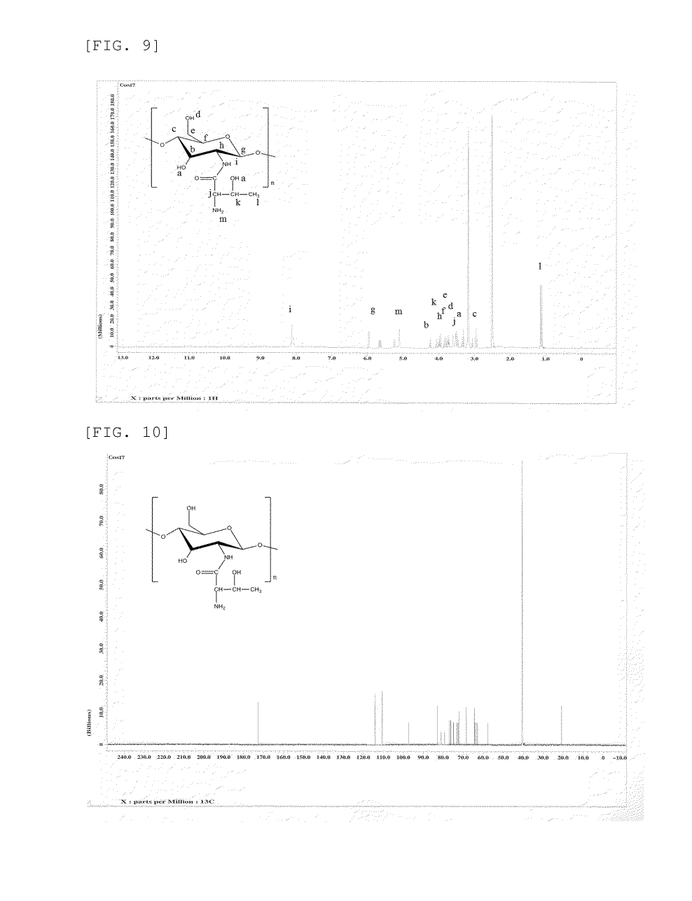

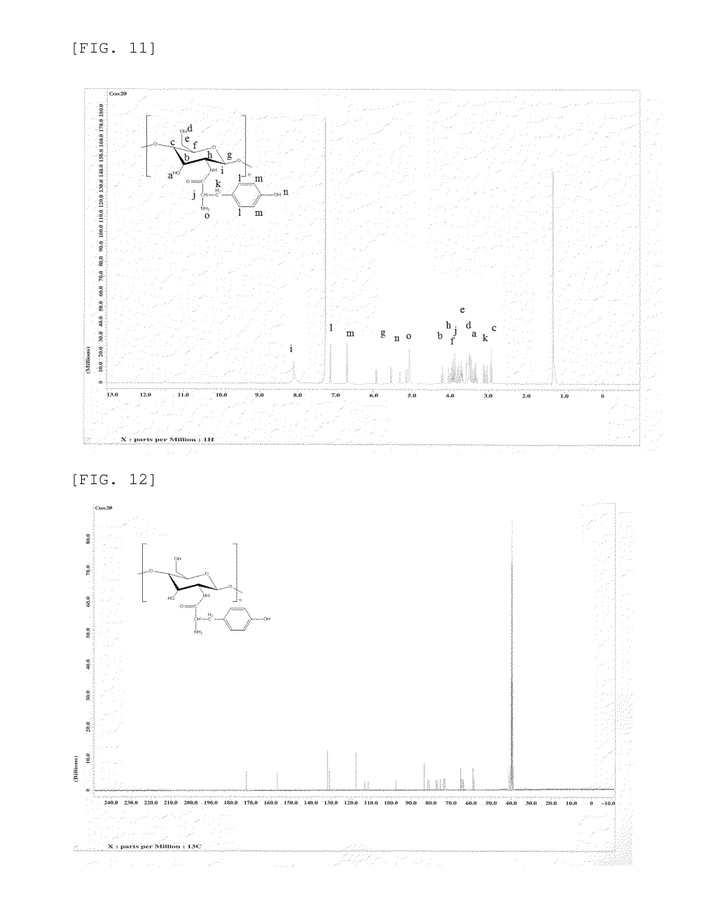

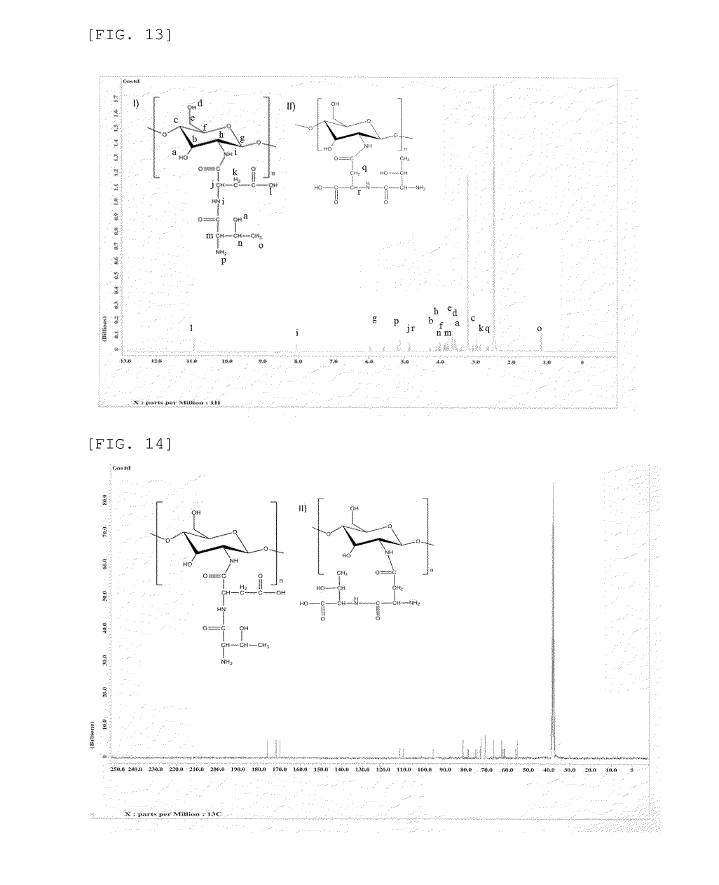

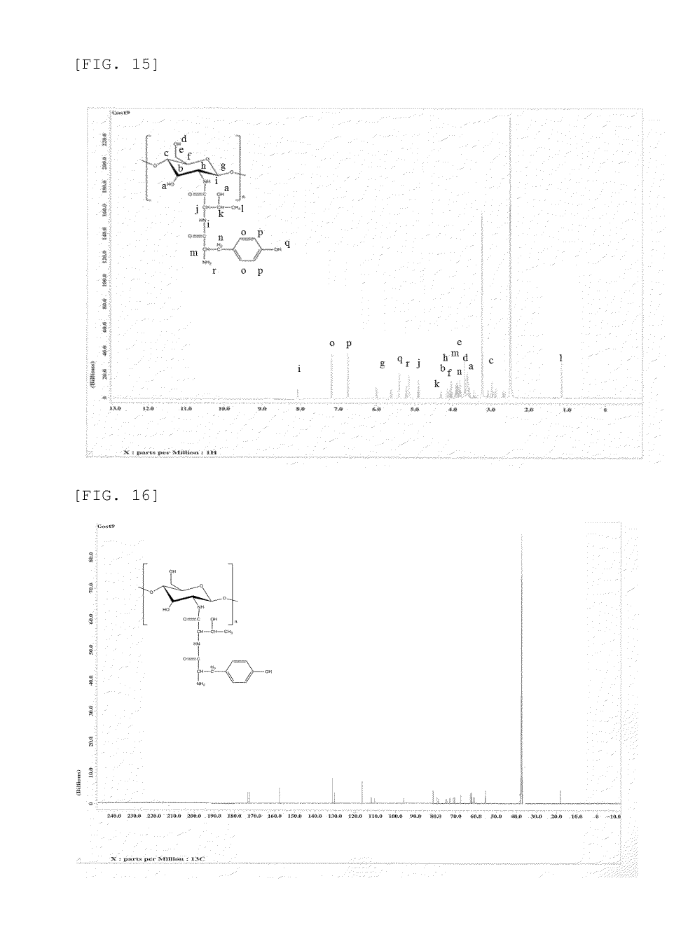

Confirmation of the synthesis was carried out by spectroscopic assays such as ¹H-NMR and ¹³C-NMR (JNM-ECP-400 NMR Spectrometer, JEOL, Japan) as well as TLC assays (see FIGS. 3 to 16). In addition to confirmation, as chitosan oligomers are polysaccharides, degree of substitution was measured by elemental analysis results for each amino acid conjugation. The following Table 1 shows types, abbreviations, yields, and degree of substitutions (DSs) of chitosan oligomer-amino acid conjugate and chitosan oligomer-dipeptide conjugate obtained from the experiments.

TABLE 1
Chitosan oligomer-amino acid/dipeptide conjugates
synthesized according to the present invention

| Compound | Abbreviation | Yield (g) | DS (%) |
|---|---|---|---|
| Chitosan oligomer-Alanine | COS-A | 0.268 | 84 |
| Chitosan oligomer-Arginine | COS-R | 0.291 | 32 |
| Chitosan oligomer-Asparagine | COS-N | 0.324 | 68 |
| Chitosan oligomer-Aspartic acid | COS-D | 0.421 | 71 |
| Chitosan oligomer-Cysteine | COS-C | 0.282 | 67 |
| Chitosan oligomer-Glutamic acid | COS-E | 0.357 | 74 |
| Chitosan oligomer-Glutamine | COS-Q | 0.405 | 76 |
| Chitosan oligomer-Glycine | COS-G | 0.217 | 91 |
| Chitosan oligomer-Eistidine | COS-H | 0.185 | 59 |
| Chitosan oligomer-Isoleucine | COS-I | 0.253 | 53 |
| Chitosan oligomer-Leucine | COS-L | 0.316 | 64 |
| Chitosan oligomer-Lysine | COS-K | 0.231 | 36 |
| Chitosan oligomer-Methionine | COS-M | 0.312 | 50 |
| Chitosan oligomer-Phenylalanine | COS-F | 0.342 | 61 |
| Chitosan oligomer-Proline | COS-P | 0.266 | 57 |
| Chitosan oligomer-Serine | COS-S | 0.413 | 87 |
| Chitosan oligomer-Threonine | COS-T | 0.354 | 81 |
| Chitosan oligomer-Tryptophan | COS-W | 0.293 | 41 |
| Chitosan oligomer-Tyrosine | COS-Y | 0.215 | 72 |
| Chitosan oligomer-Valine | COS-V | 0.227 | 89 |
| Chitosan oligomer-(Threonine- | COS-TD | 0.527 | 66 |
| Aspartic acid) | | | |
| Chitosan oligomer-(Threonine- | COS-TY | 0.485 | 68 |
| Tyrosine) | | | |

### Example 2

### Measurement of Anti-HIV Activity of Chitosam Oligomer-Amino Acid Conjugate/Dipeptide Conjugate of the Present Invention

<2-1> Measurement of Anti-HIV Activity of Chitosan Oligomer-Amino Acid Conjugates (Total 20)

The present inventors investigated a cell protecting effect and syncytia formation inhibiting effect from C8166 cell dissolution according to HIV infection after treating C8166 cell lines (Human T cell leukaemia) with the conjugates of the present invention in order to select a material having anti-HIV activity among chitosan oligomer-amino acid conjugates synthesized (prepared) through Example 1.

(1) Cell Protecting Effect Against HIV-1 Lysis Activity

In order to confirm a cell protecting effect of 20 chitosan oligomer-amino acid conjugates synthesized (prepared) through Example 1 against HIV-1 lysis activity, each of 20 conjugates according to the present invention was treated to C8166 cell lines (Human T cell leukaemia), and then the cells were infected with HIV-1. And then, the cytotoxicities were measured through MTT assay as time passed.

Since C8166 cell lines were sensitive about lysis effect of HIV-1 replication, the present test evaluated a degree of inhibiting HIV-1 replication based on the sensitivity of C8166 cells at lysis activity due to HIV-1 infection in the present experiment.

Specifically, a 300 μl aliquot containing 1×10⁵ cells was added in triplicate to the wells of a 48-well plate containing 0.5 mg/ml of the sample of chitosan oligomer-amino acid conjugate synthesized (prepared) from Example 1 in a volume of 100 μl of medium. Since then, the supernatants of HIV-1_{IIIB} were diluted in complete medium to yield sufficient cytopathicity, and a 100 μl aliquot was added to the wells. Plates were incubated for 24 h, 48 h and 72 h at 37° C. and after each respective incubation, 100 μl of 500 μg/ml MTT solution was added to each well and the plate was incubated for another 4 hours at 37° C. The formazan salt was dissolved in acidified propanol containing 50% DMSO and 4% triton X-100. Optical density was measured at 540 nm with a GENios microplate reader (Tecan, Austria GmbH, Austria). The optical density of formazan formed by untreated cells was taken as 100% of viability.

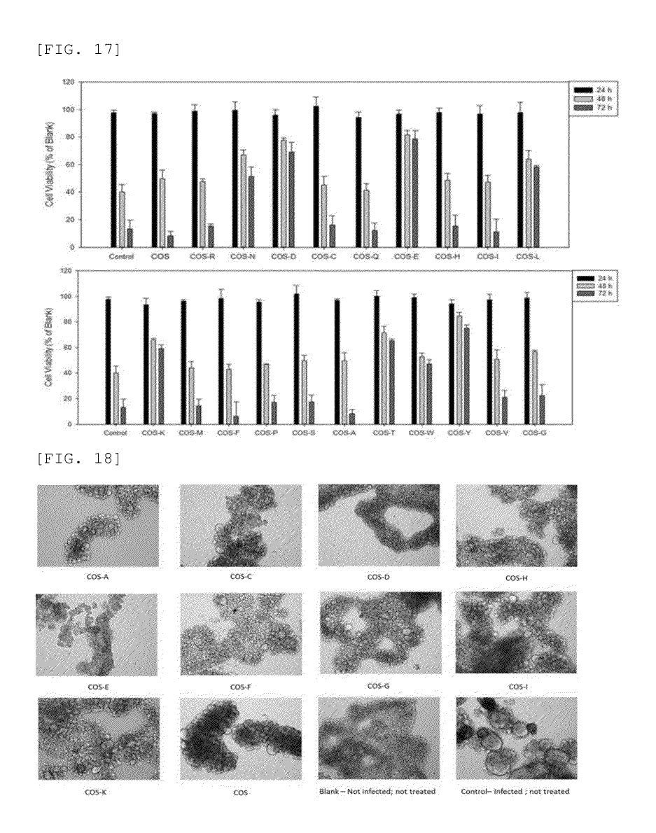

As a result, as illustrated in FIG. 17, it was confirmed that in the control group (untreated sample), cell deaths were increased time-dependently in the HIV-1-infected C8166 cells (about 90% cell kill in 5 days), while in the experimental group treated with the chitosan oligomer-amino acid conjugate of the present invention, cell deaths were inhibited. Referentially, it could be evaluated from the cell viability measurement that at the time of passing 24 hours after infection of HIV-1, the chitosan oligomer-amino acid conjugate did not have cytotoxicity.

Meanwhile, the present inventors evaluated an anti-HIV activity of each of experimental groups through a cell viability measurement at the time of passing 48 hours. It was considered that the experimental group exhibiting 70% or more of cell viability has excellent anti-HIV activity as compared with the blank group without the infection of HIV-1. As a result, COS-D, COS-E, COS-L, COS-N, COS-T and COS-Y were the active compounds which were able to keep the cells viable with the percentage of 78.8, 81.3, 70.4, 71.2, 72.3 and 84.9% respectively. Accordingly, they could be primarily selected as a material having an anti-HIV activity.

(2) Syncytia Formation Inhibiting Effect

In order to confirm effects of 20 conjugates of the chitosan oligomer-amino acid synthesized (prepared) from Example 1 described above, 20 conjugates of the present invention treated to C8166 cell lines (Human T cell leukaemia), and then the C8166 cell lines were infected with HIV-1RF (X4 tropic HIV-1 strain). Since then, a degree of syncytia formation was observed by using a microscope.

HIV-1 infection of CD4+ human T cell lines in culture results in significant cytopathic effects. Cell killing is characterized by initial formation of large, multinucleated giant cells and syncytia, followed later by destruction of single cells that swell and die. When the virus infection stimulating cell fusion was analyzed, the syncytia formation was tested in order to investigate the virus infection. HIV-1-infected cells produce high rate of a specific membrane protein to HIV-1 binding, and they conjugate and fusion with other infected cells to form a giant cell, called syncytia.

Specifically, 1×10⁵ C8166 cells in aliquots of 300 μl were seeded in triplicate to a 48-well plate containing 100 μl of serial dilutions of COS-amino acids (each of chitosan oligomer-amino acid conjugate) in complete RPMI-1640 medium with 5% FBS. After 2 hours of incubation, the cells were infected with 100 μl of stock supernatant of HIV-1_{RF} diluted in complete medium at 200 CCID₅₀ (μg/ml). The plates were incubated at 37° C. for 48 hours and the formation of syncytia was determined microscopically by using an inverted microscope (Leica Microsystems, Germany).

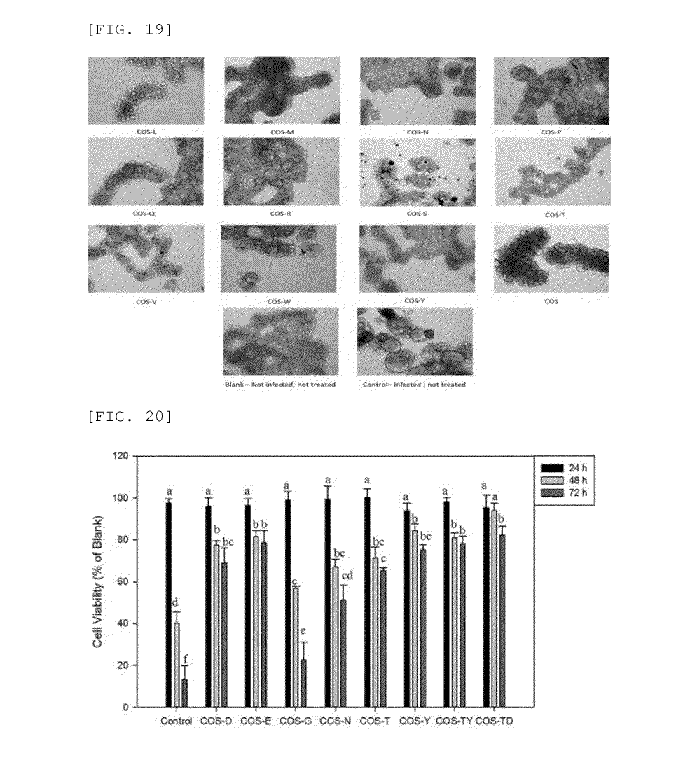

As a result, as illustrated in FIGS. 18 and 19, it was confirmed that the syncytia formation was inhibited in all the 5 conjugates (COS-D, COS-E, COS-N, COST, and COS-Y) except COS-L among 6 conjugates primarily selected as described above.

Accordingly, the present inventors selected finally COS-D, COS-E, COS-N, COST, and COS-Y as the chitosan oligomer-amino acid conjugate having high anti-HIV activity.

<2-2> Measurement of Anti-HIV Activity of Chitosan Oligomer-Dipeptide Conjugates (Total 2 Conjugates)

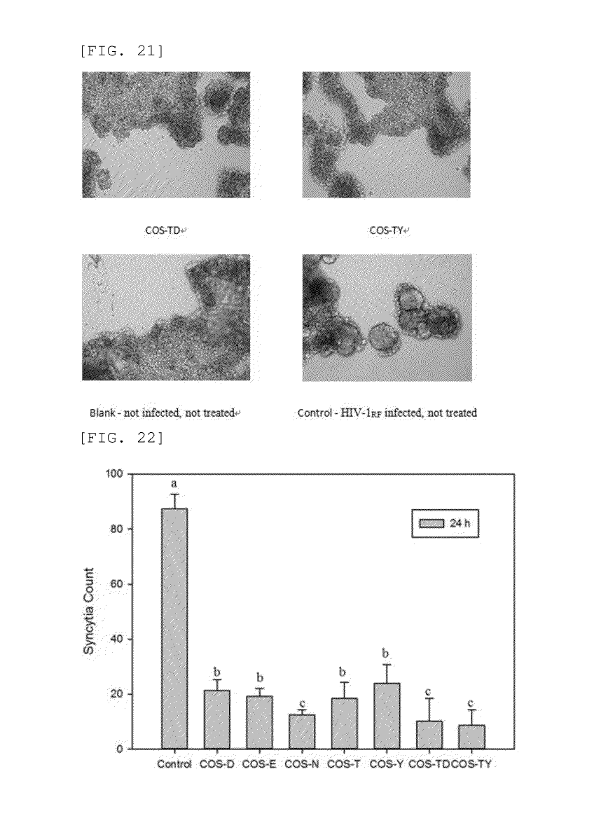

In this experiment, the anti-HIV activity of chitosan oligomer-dipeptide conjugate was analyzed by using the same method as in Example <2-1>, and the results are listed in FIGS. 20 and 21.

FIG. 20 illustrates a cell protecting effect confirmed from HIV-1 lysis activity according to the treatment of COS-TD and COS-TY of the present invention. According to FIG. 20, it could be confirmed that the experimental group treated with each of COS-TD and COS-TY exhibited high cell protecting effect. Especially, the experimental group treated with COS-TD of the present invention exhibited 94.6% of cell viability of C8166 cells at the time of passing 48 hours after HIV-1 infection. Accordingly, it could be confirmed that COS-TD exhibited highest cell protecting effect among the conjugates synthesized through Example 1.

In addition, according to FIG. 21 illustrating syncytia formation inhibiting effect according to COS-TD and COS-TY of the present invention, it was confirmed that syncytia was rarely formed in the cells treated with COS-TD and COS-TY. According to the above-described result, it could be confirmed that COS-TD and COS-TY of the present invention had excellent syncytia inhibiting effect.

<2-3> Chitosan Oligomer-Amino Acid/Dipeptide Conjugates (Total 7 Conjugates) of Present Invention Having High Anti-HIV Activity

The present inventors can select 7 conjugates as a material having high anti-HIV activity through Examples <2-1> and <2-2> described above, and the 7 conjugates are listed in the following Table 2.

TABLE 2
7 conjugates having high anti-HIV activity
according to the present invention

| Type | Structure | |
|---|---|---|
| COS-D | 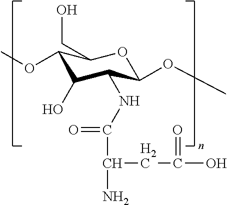 | |
| COS-E | 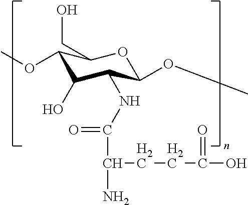 | |
| COS-N | 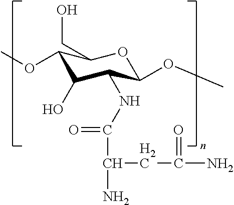 | |
| COS-T | 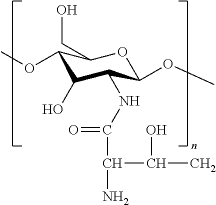 | |
| COS-Y | 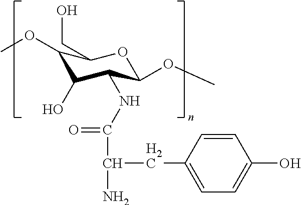 | |
| COS-TD | i 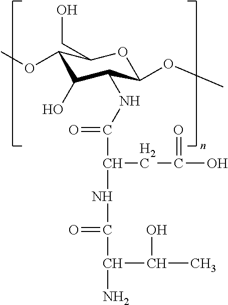 | ii 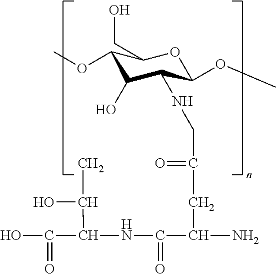 |

| | |
|---|---|
| COS-TY | 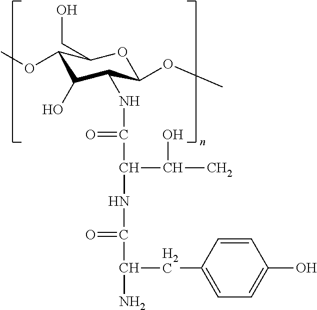 |

* n = 2~6

### Example 3

### Confirmation of Anti-HIV Activity Mechanism of Chitosan Oligomer-Amino Acid/Dipeptide Conjugate

The present inventors selected COS-D, COS-E, OCS-N, COS-T, COS-TD, and COS-TY as a material having high anti-HIV activity through Example 2 described above, and this experiment specifically confirmed the mechanism of their anti-HIV activity.

<3-1> Measurement of Syncytia Number

In Example 2, a degree of syncytia formation according to the treatment of each of COS-D, COS-E, COS-N, COS-Y, COS-TD, and COS-TY was confirmed by using a microscopy. However, in this experiment, in order to further precisely confirm the degree of syncytia formation, the number of syncytia formation according to the treatments of the above materials was calculated per each well, and then exhibited as a graph.

Basically, 1×10⁵ C8166 cells in aliquots of 300 it were seeded in triplicate to a 48-well plate containing 100 it of serial dilutions of the samples (COS-D, COS-E, COS-N, COS-T, COS-Y, COS-TD, or COS-TY) at a concentration of 0.5 mg/ml in complete RPMI-1640 medium with 5% FBS. After 2 hours of incubation, the cells were infected with 100 μl of stock supernatant of HIV-1RF diluted in complete medium at 200 CCID₅₀ (μg/ml). The plates were incubated at 37° C. for 48 hours and the formation of syncytia was determined microscopically by using an inverted microscope.

As a result, as illustrated in FIG. 22, it was confirmed that the syncytia formations in the groups treated with all the samples (COS-D, COS-E, COS-N, COS-T, COS-Y, COS-TD, or COS-TY) was significantly inhibited as compared with the control group (untreated group). Especially, it was confirmed that the syncytia formation in the groups treated with COS-TD and COS-TY was more significantly inhibited.

<3-2> Reverse Transcriptase Activity Inhibiting Effect

HIV uses reverse transcriptase (RT) for replicating a genetic material and producing a new virus. Since the role of the reverse transcriptase (RT) is important for HIV replication, the conventional reverse transcriptase (RT) inhibitors were used as a HIV treatment drug. In this experiment, a reverse transcriptase (RT) inhibiting effect was estimated as an anti-HIV activity mechanism of each of COS-D, COS-E, COS-N, COS-T, COS-Y, COS-TD, or COS-TY.

The activity of HIV-1 reverse transcriptase, isolated from virus pellet of culture supernatant was evaluated using a fluorescence RT assay kit (InvitroGen) according to the manufacturer's protocol. Briefly, 20 μl of reaction mixture containing a template/primer hybrid, poly(A)/d(T)16, and dTTP as a triphosphate substrate, was added to the wells of a microtiter plate and mixed with viral lysate containing various concentrations of samples (COS-D, COS-E, COS-N, COS-Y, COS-TD, or COS-TY [0.5 mg/ml]), which was diluted in 50 mM Tris-HCl, 20% glycerol, 2 mM DTT, pH 7.6. After incubation at 37° C. for 1 hour, the reaction was stopped by the addition of 2 μl of 200 mM EDTA to each reaction. Fluorescence intensity was measured at 480 nm (excitation) and 520 nm (emission) with a GENios® microplate reader (Tecan Austria GmbH, Austria) after the addition of 173 μl of fluorescent PicoGreen reagent prepared in TE buffer, in which the fluorescent PicoGreen reagent is selectively bound to sDNA or DNA-RNA heteroduplexes on a single stranded nucleic acid or free nucleotide. At this time, two different cell lines (HIV-1_{RF}/HIV-1_{IIIB})1 were used as the virus cell line to measure RT activity inhibiting effect. Meanwhile, 2 μM of zidovudine (AZT) that is well-known as a RT inhibitor was used as a positive control group.

As a result, as illustrated in the following Table 3 and FIG. 23, it was confirmed that COS-D, COS-Y, COS-TD, and COS-TY of the present invention effectively inhibited the reverse transcriptase (RT) activity.

TABLE 3
RT inhibition percentage and IC₅₀ value of each of
COS-D, COS-E, COS-N, COS-T, COS-Y, COS-TD, and COS-TY of the
present invention

| Compound | RT Inhibition (%) | IC₅₀ (mg/ml) |
|---|---|---|
| COS-D | 83.4 | 0.213 |
| COS-E | 32.7 | 1.036 |
| COS-N | 41.8 | 0.954 |
| COS-T | 55.3 | 0.761 |
| COS-Y | 83.5 | 0.213 |
| COS-TD | 85.1 | 0.207 |
| COS-TY | 85.8 | 0.192 |
| AZT | 82.6 | 0.081 |

*The above-described values are a mean value of independently measured values in HIV-1_{RF} and HIV-1_{IIIB}.

<3-3> Protease Inhibiting Effect

HIV-1 protease is aspartyl protease of retrovirus and an essential enzyme for HIV life cycle. Further, HIV-1 protease is used for enzyme trimming protein capsid of its progeny expressed by HIV gene. Accordingly, in the case of suppressing HIV protease, the virus replication can be blocked. In this experiment, HIV protease inhibiting effect was estimated as an anti-HIV activity mechanism of each of COS-D, COS-E, COS-N, COS-T, COS-Y, COS-TD, and COS-TY.

In order to assess the protease inhibiting effect of the compounds, commercial SensoLyte® 520 HIV-1 protease assay kit (Anaspec, Calif., USA) including fluorescence resonance energy transfer (FRET) peptide has been used according to manufacturer's directions. The amino acid sequence of FRET peptide is made by copying p17/p24 decomposition site by HIV-1 protease on a precursor Gag protein. FRET peptide is decomposed by HIV-1 protease and fluoresces.

10 μl of the sample (COS-D, COS-E, COS-N, COS-T, COS-Y, COS-TD, or COS-TY [0.5 mg/ml]) was added to 40 μl of HIV-1 protease diluents of virus and then pre-cultured at 37° C. for 15 minutes. Since then, 20 μl of substrate was added to HIV-1 protease that is pre-cultured at the same temperature for the same period and then slowly well-mixed for 30 to 60 second, completely. Since then, the fluorescence was measured at Ex/Em=340 nm/490 nm. The inhibiting rate was estimated as a relatively value to 100 of the fluorescence of the sample without an inhibitor. At this time, two different cell lines (HIV-1_{RF}/HIV-1_{IIIB}) were used as the virus cell line to measure the protease activity inhibiting effect. Meanwhile, 2 μM of saquinavir (saq) that is well-known as a protease inhibitor was used as a positive control group.

As a result, as illustrated in FIG. 24, it was confirmed that only COS-E among COS-D, COS-E, COS-N, COS-T, COS-Y, COS-TD, and COS-TY effectively inhibited HIV-1 protease. As compared with an RT inhibiting effect of Example <3-2> described above, it could be found that COS-E can protect cells from virus by HIV-2 protease inhibiting effect, not RT inhibiting effect.

Meanwhile, the present inventors examined HIV-1 protease inhibiting effect using other cell lines of HIV-1. They use HIV-1_{saq} that is a cell line having a resistance to saquinavir. Saquinavir is an anti-retrovirus medicine and is well-known as a protease inhibitor, industrially. However, since there is a variety of HIV-1 having a resistance to the medicine, other kinds of HIV-1 protease inhibitor capable of replacing saquinavir is required.

Accordingly, the present inventors tested whether or not each of COS-D, COS-E, COS-N, COS-T, COS-Y, COS-TD, and COS-TY can exhibit protease inhibiting effect on HIV-1_{saq} that is a cell line having a resistance to saquinavir. In order to assess the protease inhibiting effect of the compounds, commercial SensoLyte® 520 HIV-1 protease assay kit (Anaspec, Calif., USA) including fluorescence resonance energy transfer (FRET) peptide has been used according to manufacturer's directions as described above. Saquinavir was used as a positive control group.

As a result, as illustrated in FIG. 25, there were on a protease inhibiting effect in the positive control group, while in the case of treating COS-E of the present invention, HIV-1 protease was effectively inhibited.

Based on the above result, it could be confirmed that COS-E of the present invention can exhibit effective HIV-1 protease inhibiting effect in HIV-1_{saq} cell lines having a medicine resistant as well as the cell lines without a medicine resistant (HIV-1_{RF}/HIV-1_{IIIB}), and thus can be used for a patient exhibiting a medicine resistant to saquinavir.

<3-4> Anti-HIV Activity Mechanism of Chitosan Oligomer-Amino Acid/Dipeptide Conjugate

It was estimated that anti-HIV activity mechanism of each of COS-D, COS-E, COS-N, COS-T, COS-Y, COS-TD, and COS-TY verified in Examples <3-1> to <3-3> were not equal to each other, but had a specific property.

That is, it was confirmed that COS-D and COS-Y exhibited a strong RT inhibiting activity, COS-E exhibited a strong protease inhibiting activity, and COS-N and COS-T effectively inhibited syncytia formation, and thus had excellent effect of inhibiting cell fusion at the initial infection of virus. In addition, it could be confirmed that COS-TD and COS-TY had effects of inhibiting virus initial infection and RT at the same time.

Accordingly, the present inventors expected that by synthetically estimating the above-described results, anti-HIV activity mechanism of each of COS-D, COS-E, COS-N, COS-T, COS-Y, COS-TD, and COS-TY is as in the following Table 4.

TABLE 4
Anti-HIV activity mechanism of each of COS-D, COS-E,
COS-N, COS-T, COS-Y, COS-TD, and COS-TY

| Compound | Proposed mechanism |
|---|---|
| COS-D | RT Inhibition |
| COS-E | Protease Inhibition |
| COS-N | HIV-1 infection Inhibition |
| COS-T | HIV-1 infection Inhibition |
| COS-Y | RT Inhibition |
| COS-TD | HIV-1 infection & RT Inhibition |
| COS-TY | HIV-1 infection & RT Inhibition |

### Example 4

### Anti-HIV Activity Effect of Each of Chitosan Oligomer-Amino Acid Conjugate

The present inventors confirmed anti-HIV activity mechanism of each of COS-D, COS-E, COS-N, COS-T, and COS-Y through Example 3 described above. In this experiment, we more specifically confirmed the anti-HIV activity mechanism confirmed from Example 3 by testing the samples with various concentration.

<4-1> COS-D

It was found that COS-D of the present invention had anti-HIV activity through a reverse transcriptase (RT) inhibiting activity. In this experiment, we confirmed a reverse transcriptase inhibiting activity according COS-D with various concentrations (0.05, 0.1, 0.25, 0.5, and 1 mg/ml).

Activity of HIV-1 reverse transcriptase was measured with the same method as in Example <3-2> except that two cell lines lysates of C8166 and CEM-SS that were infected with HIV-1_{RF} were used.

As a result, as illustrated in FIG. 26, it was confirmed that COS-D of the present invention exhibited the reverse transcriptase inhibiting activity depending on the concentration. Meanwhile, there were no the reverse transcriptase inhibiting effect at the concentration of 0.05 mg/ml. Thus, it is considered that COS-D does not inactivate the reverse transcriptase function, but blocks the activity of RT enzyme by an interaction with the active site of reverse transcriptase.

<4-2> COS-Y

It was found that COS-Y of the present invention had anti-HIV activity through a reverse transcriptase (RT) inhibiting activity. In this experiment, we confirmed a reverse transcriptase inhibiting activity according COS-Y with various concentrations (0.05, 0.1, 0.25, 0.5, and 1 mg/ml).

Activity of HIV-1 reverse transcriptase was measured with the same method as in Example <4-1>.

As a result, as illustrated in FIG. 27, it was confirmed that COS-Y of the present invention exhibited the reverse transcriptase inhibiting activity depending on the concentration. However, the pattern of reverse transcriptase inhibiting activity of COS-Y is somewhat different from the pattern of reverse transcriptase inhibiting activity of COS-D. The biggest difference is that the reverse transcriptase activity was not inhibited at the concentrations of 0.05 mg/ml and 0.1 mg/ml of COS-D, but in the case of COS-Y, the reverse transcriptase activity was inhibited even at the above low concentrations. It means that COS-Y is more specifically interacted with the activation site of reverse transcriptase as compared with COS-D, and thus COS-Y is more effective for inhibiting the reverse transcriptase activity as compared with that of COS-D.

<4-3> COS-N

It was found that COS-N of the present invention had anti-HIV activity through a virus initial infection inhibiting activity. In this experiment, we confirmed a syncytia formation inhibiting activity according COS-N with various concentrations (0.2, 0.4, 0.6, 0.8, and 1.0 mg/ml).

Specifically, 1×10⁵C8166 cells in aliquots of 300 μl were seeded in triplicate to a 48-well plate containing 100 μl of serial dilutions of COS-N in complete RPMI-1640 medium with 5% FBS. After 2 hours of incubation, the cells were infected with 100 μl of stock supernatant of HIV-1_{RF} diluted in complete medium at 200 CCID₅₀ (μg/ml). The plates were incubated at 37° C. for 96 hours and the formation of syncytia was determined microscopically by using an inverted microscope (Leica Microsystems, Germany). Dextran sulfate having the syncytia formation inhibiting effect was used as a positive control group.

As a result, as illustrated in FIG. 28, it was confirmed that COS-N of the present invention inhibited the syncytia formation depending on the concentration, and more effective as compared with the positive control group.

<4-4> COS-T

It was found that COS-T of the present invention had anti-HIV activity through a virus initial infection inhibiting activity. In this experiment, we confirmed a syncytia formation inhibiting activity according COS-T with various concentrations (0.2, 0.4, 0.6, 0.8, and 1.0 mg/ml).

The syncytia formation effect was measured by using the same method as in Example <4-3>.

As a result, as illustrated in FIG. 29, it was confirmed that COS-T of the present invention inhibited the syncytia formation depending on the concentration. However, it could be confirmed that in the case of COS-T, the syncytia formation inhibiting activity was very sharply increased at the concentration of 0.5 mg/ml or more as compared with COS-N having the similar effect.

<4-5> COS-E

It was found that COS-E of the present invention had anti-HIV activity through a HIV-1 protease inhibiting activity. In this experiment, we confirmed a HIV-1 protease inhibiting effect and p27 protein production inhibiting rate in the cell culture supernatant according COS-E with various concentrations (0.05, 0.1, 0.25, 0.5, and 1.0 mg/ml).

The p24 protein of HIV plays an important role on producing HIV-1 protease as a virus core protein. Therefore, HIV-1 protease inhibiting degree can be more certainty confirmed through the measurement of p24 protein production amount.

First, the HIV-1 protease inhibiting activity was measured by using the same method as in Example <3-3> except that the protease diluents of two cell lines, C8166 and CEM-SS infected with HIV-1_{RF}.

As a result, as illustrated in FIG. 30, it was confirmed that COS-E of the present invention inhibited the protease depending on the concentration.

In order to measure p24 amount secreted from virus, H9 cells (3×10⁶ cells/ml) was cultured at 37° C. for 1 hour in the presence of HIV-1_{IIIB}. The unbound virus was removed from the cells by washing, and re-suspended in the culture medium to be 3×10⁵ cells/ml. 1 mL of aliquot was injected to 24-well plate including the medium having the same volume containing COS-E sample. In order to measure the amount of virus secreted in the medium, HIV-1 p24 antigen capture ELISA was performed according to the manufacturer's instruction using a commercial kit (Perkin-Elmer Life Sciences, Boston, Mass.).

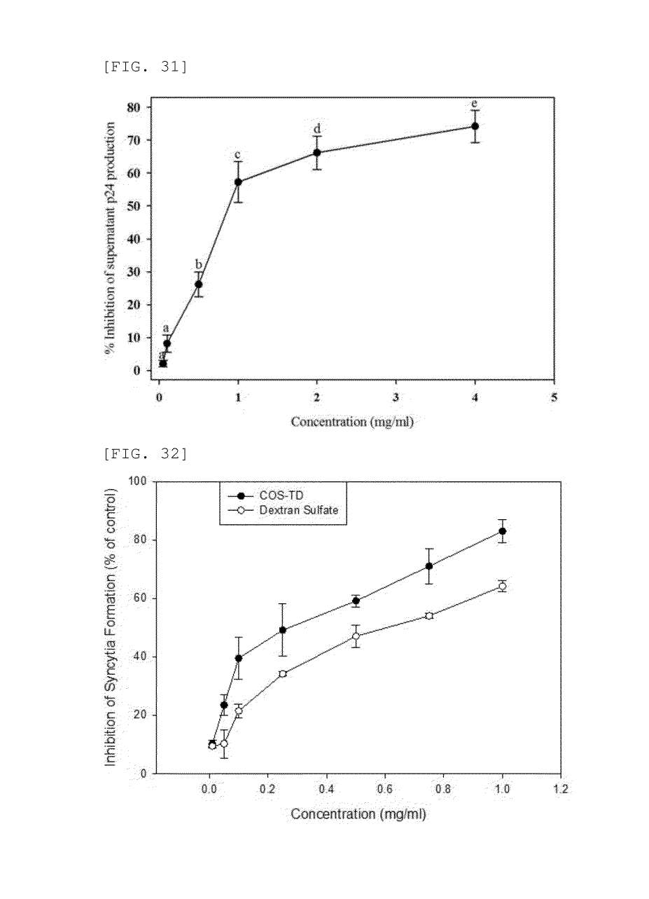

As a result, as illustrated in FIG. 31, it was confirmed that COS-E of the present invention effectively inhibited p24 secretion amount (production amount) depending on the concentration.

### Example 5

### Anti-HIV Activity Effect of Each of Chitosan Oligomer-Dipeptide Conjugates

The present inventors confirmed anti-HIV activity mechanism of each of COS-TD and COS-TY through Example 3 described above. In this experiment, we more specifically confirmed the anti-HIV activity mechanism by testing HIV-1 infection inhibiting activity, HIV-1 virus particles production inhibiting activity, p24 protein production inhibiting activity, and a gene reporter assay under presence of the samples with various concentrations.

<5-1> HIV-1 Infection Inhibiting Activity

According to Example 3 described above, since COS-TD and COS-TY of the present invention have two amino acids different from the conjugate produced by binding between chitosan oligomer and only one amino acid, COS-TD is characterized by having all two kinds of properties according to two types of amino acids. First, COS-TD and COS-TY of the present invention may have effect on the host cell membrane than the viral envelope due to a side chain, threonine (T), which was verified by a syncytia inhibiting effect of COS-TD and COS-TY (see Example <3-4>).

Therefore, in this experiment, in order to confirm whether or not the HIV-1 infection inhibiting activity was performed through interrupting an interaction between host-virus membranes by COS-TD and COS-TY, the uninfected C8166 cell lines and H9 infected with HIV-1_{IIIB} were co-cultured.

The sample (COS-TD or COS-TY) with various concentrations (0.2, 0.4, 0.6, 0.8, and 1 mg/ml) was treated to 3×10⁴ C8166 cell uninfected, and then after 2 hours, co-cultured with 3×10³ H9 cell (ratio—1:10) infected with HIV-1_{IIIB} at 37° C. After 96 hours, the formed syncytia was counted by using a microscope (100×). Dextran sulfate having the syncytia formation inhibiting effect was used as a positive control group.

As a result, as illustrated in FIG. 33, it was confirmed that in the case of treating with each of COS-TD and COS-TY of the present invention, the syncytia formation was inhibited depending on the treated concentration, and more effective as compared with the positive control group.

<5-2> HIV-1 Virus Particle Production Inhibiting Activity

Since HIV-1 virus requires virus particles in order to form new virus itself by inserting in a host, HIV-1 virus infection can be generally diagnosed through detecting virus particles. Therefore, in this experiment, in order to estimate whether or not COS-TD and COS-TY can successfully inhibit HIV-1 infection, the amount of produced p24 antigen, and HIV-1 gene expression such as Vif, Env, and Gag were analyzed.

(1) p24 Antigen Production Amount Analysis

The amount of p24 protein in a cell according to COS-TD and COS-TY of the present invention was measured through a western blot analysis. First, each of COS-TD and COS-TY with various concentrations (0.01, 0.05, 0.1, 0.25, 0.5, and 1 mg/ml) was treated to H9 cells infected with HIV-1_{IIIB}, and then cultured for 4 days. The total cultured cells were lysised in RIPA buffer solution (Sigma-Aldrich Corp., St. Louis, USA), centrifuged, and then the total protein amount of cell lysate was measured by using Lowry method (BioRad Laboratories, Hercules, Calif.). The aliquot supernatant including the same volume of protein (15 μg) was subjected to 10% or 12% SDS-PAGE gel, transferred on a nitrocellulose membrane (Amersham Pharmacia Biotech., England, UK), and blocked in Tris buffered saline containing 0.1% (v/v) Tween-20 (TBS-T) and 5% skim milk powder for at least 1 hour. The membrane was hybridized with mouse anti-p24 monoclonal antibody (Santa Cruz Biotechnology Inc., CA, USA). All the primary monoclonal antibodies were diluted with TBS-T at a ratio of 1:1000. The antibody was detected with Horseradish peroxidase (HRP)-conjugated anti-mouse IgG secondary antibody for 1 hour. The immune reactive protein was detected by using a chemical fluorescence ECL detecting kit (Amersham Pharmacia Biosciences, England, UK) according to the manufacturer's instruction. The western blot bands were visualized by LAS3000® chemiluminescence (Fujifilm Life Science, Tokyo, Japan).

As a result, as illustrated in FIG. 34, it could be confirmed that in the case of the experimental groups treated each of COS-TD and COS-TY of the present invention, p24 antigen production was inhibited depending on the treatment concentrations.

(2) HIV-1 Gene (Vif, Env, Gag) Expression Analysis

The amount of intracellular HIV-1 gene (Vif, Env, Gag) expression according to the treatment of COS-TD and COS-TY of the present invention was measured by using a RT-PCR. First, each of COS-TD and COS-TY with various concentrations (0.05, 0.1, 0.5, and 1 mg/ml) was treated to H9 cells infected with HIV-1_{IIIB}, and then cultured for 4 days. RNA in the total cells cultured was isolated by using Trizol reagent (Invitrogen Co., CA, USA). 2 μg of isolated RNA was reverse-transcripted into cDNA using an oligo-(dT) primer (Promega, Madison, Wis., USA). The target cDNA was amplified by using forward and reverse primer sequences (see the following Tables 5 to 7). A PCR product was isolated by subjecting an electrophoresis on 1.5% agarose gel at 100 V for 10 minutes. The gel was stained with 1 mg/ml of EtBr, and then photographed under UV illumination by using AlphaEas® gel image analysis software (Alpha Innotech., San Leandro, Calif., USA).

As a result, as illustrated in FIG. 35, it was confirmed that in the case of the experimental group treated with each of COS-TD and Cos-TY of the present invention, all the Vif, Env, and Gag genes expressions were reduced depending on the treatment concentrations, and especially, the Env gene expression was significantly inhibited as the treatment concentrations of COS-TD and COS-TY were increased.

TABLE 5
Reagent and Reaction combination used for RT-PCR test

| PCR Chemical | Amount used | Stock | |
|---|---|---|---|
| MMLV reverse transcriptase | 0.5 μl | 200 | U/μl |
| dNTP mixture | 1 μl | 10 | mM |
| DTT | 1 μl | 100 | mM |
| 5X reaction buffer | 4 μl | | |
| RNase inhibitor | 0.5 μl | 80 | U/μl |

TABLE 6
PT-PCR condition

| Temperature | Time |
|---|---|
| 42° C. | 1 h 30 min |
| 95° C. | 5 min |
| 4° C. | ∞ |

TABLE 7
HIV-1 genes primers sequences

| Amplified | |
|---|---|
| gene | Primer Sequence |
| HIV-1 Vif | Sense: 5′-GAGTCAACGGATTTGGTCGT-3′ |
| | Antisense: 5′-GACAAGCTTCCCGTTCTCAG-3′ |
| Human | Sense: 5′-GGTACAGTGCAGGGAAAGA-3 |
| GAPDH | Antisense: 5′-CTTGCCACACAATCATCACC-3′ |
| HIV-1 gag | Sense: 5′-GGCACATCAAGCAGCCATGC-3′ |
| | Antisense: 5′-TAGTTCCTGCTATGTCACTTCC-3′ |
| HIV-1 env | Sense: 5′-CCTCAGCCATTACACAGGCC-3′ |
| | Antisense: 5′-CCTTGGTGGGTGCTACTCCT-3′ |

<5-3> Measurement of Cell Syncytia Inhibiting Effect of COS-TD and COS-TY Through Co-Culture Method

To ensure the host cell membrane receptor interaction of COS-TD and COS-TY a co-cultivation assay was carried out. Two cell lines, 8E5 and MOLT-4 have specific mutated host cell membrane receptors of CXCR4 and CD4+ respectively which when combined forms syncyta in the absence of HIV-1 infection and mimic the cell death of HIV-1 lytic effects due to their viral particle genome integration. Therefore, this co-cultivation assay is a reliable method in order to obtain detailed information whether the COS-TD and COS-TY interrupt host cell membrane interaction without the viral deformation.

Uninfected 1×10⁴ MOLT-4 cells were co-cultured with 8E5 cells (clonal cell line derived from HIV-infected CEM, and chronically integrated with HIV-1 viral particles) in the presence of COS-TD and COS-TY (0.5 mg/ml). The number of syncytia formed with time was determined. Mock was an experimental group without the sample and AZT-Saq treated with AZT and saquinavir at the same time was used as a positive control.

As a result, as illustrated in FIG. 36, it was confirmed that in the case of the experimental group treated with each of COS-TD and COS-TY of the present invention, the number of syncytia formed with time even when passing many hours was not increased, while in the case of the MOCK without the sample, the number of syncytia formed was significantly increased at the time of passing 40 hours and Mock group does not include data after 72 h because the all cells died of HIV-1 lytic effects. In addition, the cos-TD and COS-TY groups of the present invention exhibited high syncytia formation inhibiting effect as compared with the control group. From the above-described results, it could be verified that COS-TD and COS-TY of the present invention interacts with the host cell membrane regardless the interaction with virus.

<5-4> Effect of COS-TD and COS-TY on Post-Infection of HIV

In order to confirm the efficiency of COS-TD and COS-TY against post-infection of HIV, in this experiment, the cells were infected with HIV-1, and then tested by delayed addition of the samples (COS-TD or COS-TY). This method was carried out in order to gain information whether COS-TD and COS-TY were able to interrupt already infected cells by reducing the amount of newly formed viruses.

Uninfected CEM-SS cells were cultured into individual wells of a 48-well microtiter plate at a density of 3×10⁴ cells/well in 400 μl of medium. Diluted HIV-1_{IIIB} and HIV-1Saq stock supernatants (100 μl) were added to appropriate wells to yield a final M.O.I of 1.0 (CCID₅₀). At various times after the addition of virus, a 100 μl aliquot of COS conjugates (COS-TD and COS-TY) at the concentrations of 0.5 mg/ml was added to multiple wells. After a total of 4 days incubation, in order to measure the amount of p24 production, HIV-1 p24 antigen capture ELISA was performed using the commercial kit (Perkin-Elmer Life Sciences, Boston, Mass.) according to the manufacturer's instruction.

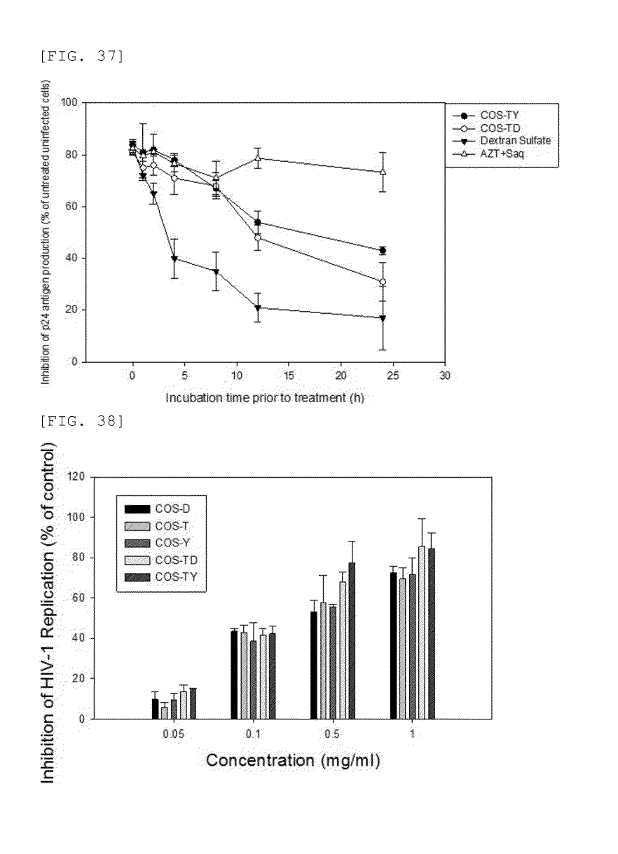

As a result, as illustrated in FIG. 37, it was confirmed that in the case of the experimental group treated with each of COS-TD and COS-TY of the present invention, p24 antigen production could be inhibited until passing 6 hours, but p24 production inhibiting activity was reduced as time passes after treating. It was considered that COS-TD and COS-TY of the present invention did not interact with the host cells as the number of the infected host cells was increased because the infected host cells continuously produced p24 as a new virion.

<5-5> Gene Reporter Assay

In this experiment, in order to estimate HIV-1 host genome replication inhibiting activities of COS-TD and COS-TY, a luciferase and beta-galactosidase gene reporter assay was conducted by using TZM-b1 cell.

Briefly, one day before infection, 5×10⁴ TZM-d1 cells were seeded in a 24-well flat, and added with 100 μl of HIV-1_{IIIB} supernatant (100 CCID₅₀ (1 μg/ml)) to infect 50% of the cells. Since then, each of the samples (COS-D, COS-T, COS-Y, COS-TD, and COS-TY) with various concentrations (0.05, 0.1, 0.5, and 1 mg/ml) was treated to the cells and then cultured for 48 hours. After incubation, cells were washed with cold PBS and lysed in 300 μl of lysis buffer (Promega Corp., Madison, Wis.). 20 μl of cell lysate was mixed with 100 μl of luciferase substrate (Promega) and light emission was measured for 10 seconds in a luminometer (Tecan Austria GmbH, Austria). In case of β-galactosidase assay, 100 μl of X-gal substrate was mixed with cell lysate in order to stain the HIV-1 replicated cells and light absorption was measured at the optical density of 550 nm.

As a result, as illustrated in FIG. 38, it was confirmed that each of COS-D, COS-T, COS-Y, COS-TD, and COS-TY of the present invention inhibited the HIV-1 replication depending on the concentration, and among them, COS-TD and COS-TY were more effective.

Consequently, it can be found that COS-TD and COS-TY can block HIV-1 binds to host cells through an interaction with a host cell, and at the same time, have an effect on inhibiting the binding between HIV-1 and a host cells through inhibiting HIV-1 reverse transcriptase activity. Accordingly, it can be suggested that COS-TD and COS-TY are usefully used as an anti-HIV medicine material.

As set forth above, according to exemplary embodiments of the invention, a new compound has an excellent anti-HIV effect through an activity of inhibiting a HIV initial infection by interrupting an interaction between host-virus membranes, and also activities of inhibiting reverse transcriptase and protease of HIV. Accordingly, the composition including the new compound as an effective component can exhibit excellent HIV-preventing or treating/improving effects, so that it can be usefully used as a functional medicine composition and food composition. Especially, the new compound according to the present invention is a conjugate synthesized through conjugating chitooligosaccharides derived from a natural material with amino acids or dipeptides. Since the new compound has stability without cytotoxicity, it has advantages that the composition of the present invention including the new compound as an effective component is stable even when it is used for a long period of time.

While the present invention has been shown and described in connection with the exemplary embodiments, it will be apparent to those skilled in the art that modifications and variations can be made without departing from the spirit and scope of the invention as defined by the appended claims.

What is claimed is:

1. A method of preventing or treating human immunodeficiency virus (HIV) infection comprising administering to a subject in need thereof a pharmaceutical composition comprising a compound represented by the following Chemical Formula 1 as an effective component: 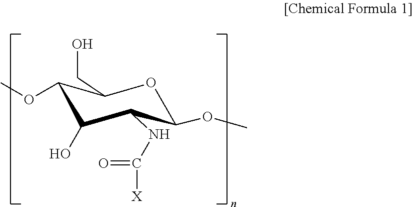
(where,
X is selected from the group consisting of Chemical Structures listed in the following Table; and 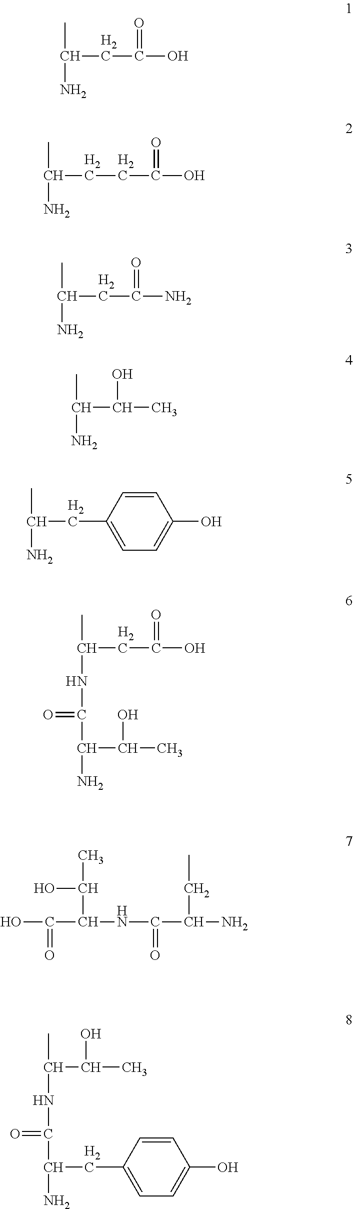
n is an integer of 2 to 6.)

2. The method of claim 1, wherein the composition including the compound represented by Chemical Formula 1 (here, x is Chemical Structure 1 or 5) as an effective component has an anti-HIV effect through an activity of inhibiting reverse transcriptase of HIV.

3. The method of claim 1, wherein the composition including the compound represented by Chemical Formula 1 (here, x is Chemical Structure 3 or 4) as an effective component has an anti-HIV effect through an activity of inhibiting a HIV infection by interrupting an interaction between host-virus membranes.

4. The method of claim 1, wherein the composition including the compound represented by Chemical Formula 1 (here, x is Chemical Structure 2) as an effective component has an anti-HIV effect through an activity of inhibiting protease of HIV.

5. The method of claim 1, wherein the composition including the compound represented by Chemical Formula 1 (here, x is one selected from Chemical Structures 6 to 8) as an effective component has an anti-HIV effect through activities of inhibiting reverse transcriptase of HIV and a HIV infection by interrupting an interaction between host-virus membranes.

6. The method of claim 1, wherein the compound represented by Chemical Formula 1 described above is included in a concentration of 0.01 to 10 mg/ml in the composition.

7. A HIV inhibitor comprising the composition according to claim 1 as an effective component.
